(19) United States
(12) Patent Application Publication
(10) Pub. No.: US 2018/0116522 A1
(43) Pub. Date: May 3, 2018

(54) METHODS, SYSTEMS AND DEVICES FOR CREATING A BLOOD FLOW PATHWAY TO TREAT A PATIENT

(71) Applicant: Rox Medical, Inc., San Clemente, CA (US)

(72) Inventors: Rodney Brenneman, San Juan Capistrano, CA (US); Peter Balmforth, Oslo (NO); Paul A. Sobotka, West St. Paul, MN (US); J. Christopher Flaherty, Auburndale, FL (US)

(21) Appl. No.: 15/858,997

(22) Filed: Dec. 29, 2017

Related U.S. Application Data
(63) Continuation of application No. PCT/US2016/041783, filed on Jul. 11, 2016.
(60) Provisional application No. 62/191,234, filed on Jul. 10, 2015.
(60) Provisional application No. 62/269,428, filed on Dec. 18, 2015.

(51) Int. Cl. A61B 5/00; A61F 2/06; A61F 2/24

(52) U.S. Cl. CPC A61B 5/0044; A61B 5/6862; A61B 5/021; A61F 2/24; A61F 2/06

(57) ABSTRACT

Provided is a system for creating an arteriovenous flow pathway in a patient, comprising: a vessel-to-vessel guidewire; a needle delivery device constructed and arranged to place the vessel-to-vessel guidewire from a starting vessel to a target vessel; a flow creation device constructed and arranged to be advanced over the vessel-to-vessel guidewire and to create a flow pathway between the starting vessel and the target vessel; and a stent for positioning in a vessel. At least one of the starting vessel or the target vessel is a vein, and the other of the starting vessel or the target vessel is an artery. The stent is constructed and arranged to be positioned in the vein and to at least one of prevent or treat venous stenosis.

### CROSS-REFERENCE TO RELATED APPLICATIONS

This application is a continuation of PCT Application No. PCT/US2016/41783 (Attorney Docket No. 29919-714.601), filed Jul. 11, 2016, which claims the benefit of U.S. Provisional Application Ser. No. 62/191,234 (Attorney Docket Number 29919-714.101), filed Jul. 10, 2015, and U.S. Provisional Application 62/269,428 (Attorney Docket Number 29919-714.102), filed Dec. 18, 2015, the content of each of which is incorporated herein by reference in its entirety for all purposes.

This application is also related to, but does not claim priority to, the following applications: U.S. Pat. No. 8,641,747, entitled “Devices for Arterio-Venous Fistula Creation”, filed Jun. 13, 2005; U.S. Pat. No. 8,016,782, entitled “Methods for Providing Oxygenated blood to Venous Circulation”, filed Jun. 13, 2005; U.S. Non-Provisional application Ser. No. 14/620,007, entitled “Device and Method for Establishing an Artificial Arterio-Venous Fistula”, filed Feb. 11, 2015; U.S. Non-Provisional application Ser. No. 14/868,652, entitled “Devices, Systems, and Methods for Creation of a Peripherally Located Fistula”, filed Sep. 29, 2015; U.S. Non-Provisional application Ser. No. 13/748,397, entitled “Devices, Systems, and Methods for Peripheral Arteriovenous Fistula Creation”, filed Jan. 23, 2013; U.S. Non-Provisional application Ser. No. 14/587,314, entitled “Device and Method for Establishing an Artificial Arteriovenous Fistula”, filed Dec. 31, 2014; U.S. Non-Provisional application Ser. No. 12/905,412, entitled “Devices, Systems, and Methods for Enhanced Visualization of the Anatomy of a Patient”, filed Oct. 15, 2010; and U.S. Non-Provisional application Ser. No. 14/668,733, entitled “Methods, Systems and Devices for Treating Hypertension”, filed Mar. 25, 2015; U.S. Non-Provisional application Ser. No. 14/766,125, entitled”, entitled “Methods, Systems and Devices for Treating Cardiac Arrhythmias”, filed Aug. 5, 2015; U.S. Non-Provisional application Ser. No. 14/970,217, entitled “Methods, Systems and Devices for Treating Erectile Dysfunction”, filed Dec. 15, 2015; and U.S. Non-Provisional application Ser. No. 15/173,505, entitled “Methods, Systems and Devices for Treating Orthostatic Intolerance”, filed Jun. 3, 2016; the content of each of which is incorporated herein by reference in its entirety.

### TECHNICAL FIELD

The embodiments disclosed herein relate generally to systems, devices and methods for treating a patient, particularly a patient afflicted with angina, mitral valve regurgitation and/or venous stenosis.

### BACKGROUND

Angina is chest pain or other discomfort caused when the heart muscle doesn't get enough oxygen-rich blood. Angina is a symptom of an underlying heart problem, usually coronary heart disease (CHD). Angina pectoris (stable angina) typically occurs during time of increased cardiac workload, such as exercise or stress, when the heart doesn't get enough oxygen (ischemia). Onset of angina pectoris is usually predictable and managed with rest and/or taking of nitroglycerin (GTN). Nitroglycerin relaxes the coronary arteries and other blood vessels, easing the heart's workload and increasing the heart's blood supply.

Mitral regurgitation, also referred to as mitral valve regurgitation, mitral insufficiency or mitral incompetence, is a condition in which the heart's mitral valve doesn't close tightly, allowing blood to flow backward in the heart. As a result, blood can't move through the heart or to the rest of the body as efficiently, making the patient feel tired or out of breath. In some cases, heart surgery is performed to repair or replace the mitral valve. Left untreated, severe mitral valve regurgitation can cause heart failure or an arrhythmia.

Venous stenosis is a narrowing of a segment of vein. Deep vein thrombosis is a form of stenosis in which the narrowing is due to a manifestation of venous thromboembolism. Treatment of venous stenosis can include implanting a stent in the narrowed segment.

There is a need for improvement systems, devices and methods for treating angina, mitral valve regurgitation and venous stenosis.

### SUMMARY

According to one aspect of the technology, a method of treating angina in a patient comprises selecting a patient exhibiting angina and creating a flow pathway between a first vascular location and a second vascular location. The first vascular location comprises a source of arterial blood and the second vascular location comprises a source of venous blood. The method is constructed and arranged to treat angina.

According to another aspect of the technology, a method of treating mitral regurgitation in a patient comprises selecting a patient exhibiting mitral regurgitation and creating a flow pathway between a first vascular location and a second vascular location. The first vascular location comprises a source of arterial blood and the second vascular location comprises a source of venous blood. The method is constructed and arranged to treat mitral regurgitation.

In some embodiments, the method is constructed and arranged to decrease angina in a patient.

In some embodiments, the method is constructed and arranged to eliminate angina in a patient.

In some embodiments, the method is constructed and arranged to cause an effect selected from the group consisting of: reduce cardiac work; reduce arterial pressure; reduce left ventricular pre-load; shift blood volume toward the venous system; increase systemic oxygenation; increase venous oxygenation; increase delivery of oxygen to tissue; and combinations thereof.

In some embodiments, the method is constructed and arranged to reduce a persistent need for at least one arrhythmia medication, such as to reduce the need for glyceryl trinitrate.

In some embodiments, the method is constructed and arranged to decrease mitral regurgitation in a patient.

In some embodiments, the method is constructed and arranged to eliminate mitral regurgitation in a patient.

In some embodiments, the method is constructed and arranged to treat a form of mitral regurgitation selected from the group consisting of: Type I mitral regurgitation; Type II mitral regurgitation; Type III mitral regurgitation; mild mitral regurgitation; moderate mitral regurgitation; moderate to severe mitral regurgitation; severe mitral regurgitation; and combinations thereof.

In some embodiments, the method is further constructed and arranged to increase the flow of blood to the right side of the heart. The increased flow of blood to the right side of the heart can increase cardiac output.

In some embodiments, the method is further constructed and arranged to increase cardiac output.

In some embodiments, the method is further constructed and arranged to reduce arterial hypertension.

In some embodiments, the method is further constructed and arranged to treat a patient disease or disorder selected from the group consisting of: hypertension; arterial hypertension; chronic obstructive pulmonary disease; congestive heart failure; lung fibrosis; adult respiratory distress syndrome; lymphangioleiomyomatosis; pulmonary hypertension; sleep apnea such as sleep apnea due to hypoxemia or hypertension; arrhythmia; erectile dysfunction; orthostatic intolerance; and combinations thereof.

In some embodiments, the method is constructed and arranged to cause a physiologic change in the patient selected from the group consisting of: reduced angina; reduced mitral regurgitation; increased oxygen delivery by the arterial system; increased blood volume; increased proportion of blood flow to the descending aorta; increased blood flow to the kidneys; increased blood flow outside the kidneys; increased cardiac output; and combinations thereof.

In some embodiments, the method is constructed and arranged to cause one or more of: a decrease in systemic vascular resistance; a decrease in blood pressure; an increase in cardiac output; an increase in right atrial pressure; an atrial natriuretic peptide (ANP) release; vasodilation; an increase in right atrial filling; a Bainbridge reflex; an increase in heart rate; peripheral sympatho-inhibition; activation of venous baroreceptors; activation of pulmonary arterial mechanoreceptors; an increase in venous oxygenation; an increase in pulmonary blood flow; an increase in arterial compliance; a decrease in a reflected pulse wave; a decrease in effective arterial volume; an increase in oxygen delivery to tissue; a decrease in chemoreceptor activity; a decrease in sympatho-excitation; sodium and/or water retention; reduced renal ischemia; and combinations thereof.

In some embodiments, creating the flow pathway comprises a procedure selected from the group consisting of: dilating tissue within, adjacent to, near to or otherwise proximate (“proximate” herein) the flow pathway with a balloon; applying energy to tissue proximate the flow pathway such as RF energy applied to tissue; and combinations thereof.

In some embodiments, the flow pathway comprises a fistula.

In some embodiments, the flow pathway comprises a flow rate of at least 400 ml/min and/or the flow pathway comprises a flow rate of less than or equal to 1500 ml/min.

In some embodiments, the first vascular location comprises an iliac artery. The second vascular location can comprise an iliac vein.

In some embodiments, the first vascular location comprises an artery selected from the group consisting of: aorta; axillary; brachial; ulnar; radial; profundal; femoral; iliac; popliteal; and carotid. The second vascular location can comprise a vein.

In some embodiments, the second vascular location comprises a vein selected from the group consisting of: inferior vena cava; saphenous; femoral; iliac; popliteal; brachial; basilic; cephalic; medial forearm; medial cubital; axillary; and jugular. The first vascular location can comprise an artery.

In some embodiments, the first vascular location comprises a chamber of the heart. The first vascular location can comprise the left atrium and the second vascular location can comprise the right atrium. The first vascular location can comprise the left ventricle and the second vascular location can comprise the coronary sinus.

In some embodiments, the first vascular location comprises the aorta and the second vascular location comprises a vein, and the flow pathway comprises a graft positioned between the aorta and the vein.

In some embodiments, the flow pathway comprises a diameter of at least 2.5 mm. The flow pathway can comprise a diameter of at least 3.0 mm. The flow pathway can comprise a diameter of less than or equal to 6.0 mm. The flow pathway can comprise a diameter of less than 5.0 mm. The flow pathway can comprise a diameter of less than 4.0 mm.

In some embodiments, the flow pathway comprises a diameter based on a patient parameter. The patient parameter can comprise a parameter determined prior to the creation of the flow pathway. The patient parameter can comprise a parameter determined during the flow pathway creation procedure. The patient parameter can comprise a parameter measured after the creation of the flow pathway, and the method can further comprise modifying the flow pathway diameter based on the measured patient parameter. The patient parameter can comprise a parameter selected from the group consisting of: angina level; mitral regurgitation severity; mitral regurgitation type; cardiac output; blood pressure; flow rate; tilt table test result; ankle brachial index test result; venous insufficiency level; peripheral vascular resistance; shunt flow; pulmonary capillary wedge pressure; right atrial pressure; pulmonary pressure; left atrial pressure; arterial oxygenation; venous oxygenation; and combinations thereof.

In some embodiments, the method further comprises creating a second flow pathway between a third vascular location and a fourth vascular location. The second flow pathway can comprise a fistula. The second flow pathway can be created at least twenty four hours after the creation of the first flow pathway. The first vascular location can comprise an artery and the third vascular location can comprise the same artery. The second vascular location can comprise a vein and the fourth vascular location can comprise the same vein. The first vascular location can comprise the iliac artery in the right leg of the patient and the third vascular location can comprise the iliac artery in the left leg of the patient. The cumulative flow rate of the first flow pathway and the second flow pathway can comprise a flow rate of at least 400 ml/min. The cumulative flow rate of the first flow pathway and the second flow pathway can comprise a flow rate of less than or equal to 1500 ml/min. The method can further comprise creating multiple flow pathways comprising at least a third flow pathway. The cumulative flow rate of the multiple flow pathways can comprise a flow rate of at least 400 ml/min. The cumulative flow rate of the multiple flow pathways can comprise a flow rate of less than or equal to 1500 ml/min.

In some embodiments, the method further comprises placing an implant proximate the flow pathway. The implant can comprise an anastomotic clip placed between the first vascular location and the second vascular location. The anastomotic clip can comprise at least a covered portion. The implant can comprise a component selected from the group consisting of: anastomotic clip; suture; staple; adhesive; and combinations thereof. The implant can comprise at least a biodegradable portion.

In some embodiments, the method further comprises dilating the flow pathway. The dilating the flow pathway can comprise dilating the flow pathway by inflating a balloon in the flow pathway.

In some embodiments, the method further comprises modifying the flow pathway. The modifying the flow pathway can comprise dilating at least a portion of the flow pathway. The method can further comprise placing an anastomotic clip in the flow pathway and the modifying the flow pathway can be performed after placement of the anastomotic clip. The modifying the flow pathway can comprise delivering energy to the flow pathway. The energy delivered can comprise radiofrequency (RF) energy. The modifying the flow pathway can be performed at least 24 hours after the creating of the flow pathway. The modifying the flow pathway can comprise modifying a flow pathway parameter selected from the group consisting of: flow pathway cross sectional diameter; flow pathway average cross sectional diameter; flow pathway flow rate; flow pathway average flow rate; diastolic pressure after flow pathway creation; diastolic pressure change after flow pathway creation; systolic pressure after flow pathway creation; systolic pressure change after flow pathway creation; ratio of diastolic to systolic pressure after flow pathway creation; difference between diastolic pressure and systolic pressure after flow pathway creation; and combinations thereof. The modifying the flow pathway can be constructed and arranged to perform a function selected from the group consisting of: increasing flow through the flow pathway; decreasing flow through the flow pathway; increasing the diameter of at least a segment of the flow pathway; decreasing the diameter of at least a segment of the flow pathway; removing tissue proximate the flow pathway; stenting a stenosis proximate the flow pathway; stenting a venous stenosis; delivering an agent to the flow pathway; delivering an agent to tissue proximate the flow pathway; delivering an agent to venous and/or arterial wall tissue proximate the flow pathway; blocking a sidebranch proximate the flow pathway; and combinations thereof.

In some embodiments, the method further comprises performing a flow pathway assessment procedure. The flow pathway assessment procedure can comprise an anatomical measurement procedure. The anatomical measurement procedure can comprise performing a measurement selected from the group consisting of: a flow pathway diameter measurement; a flow pathway length measurement; a measurement of the distance between an artery and vein comprising the flow pathway; a measurement of the distance between the flow pathway and a vessel sidebranch; and combinations thereof. The flow pathway assessment procedure can comprise measuring flow at least one of within or proximate the flow pathway. The flow pathway assessment procedure can comprise measuring a flow selected from the group consisting of: flow through the flow pathway; flow in a vessel segment proximate the flow pathway; flow measured using Doppler ultrasound; flow measured using angiographic techniques; and combinations thereof. The flow pathway assessment procedure can comprise an assessment of a patient physiologic condition. The patient physiologic condition can comprise a condition selected from the group consisting of: angina level; mitral regurgitation type; mitral regurgitation severity; orthostatic intolerance level; dizziness level; lightheadedness level; syncope events; nausea level; fatigue level; tremor state; breathing state; swallowing ability; headache level; visual disturbance level; sweating level; pallor state; cardiac output; blood pressure such as systolic and/or diastolic blood pressure; respiration; a blood gas parameter; blood flow such as blood flow through a vein or artery within and/or otherwise proximate the right side of the heart; vascular resistance; pulmonary resistance; average clotting time assessment; serum creatinine level assessment; and combinations thereof.

In some embodiments, the method further comprises applying an agent to a vein wall proximate the flow pathway. The agent can be applied to reduce venous stenosis formation. The agent can be applied in the vein downstream of the flow pathway. The agent can be applied at a time selected from the group consisting of: prior to creation of flow pathway; during creation of flow pathway; after creation of flow pathway; and combinations thereof. The agent can be applied to the vein wall at a location downstream from the flow pathway. The agent can comprise an agent selected from the group consisting of: anti-proliferative agent; a chemotherapeutic agent; paclitaxel; an mTOR inhibitor; Sirolimus; Zotarolimus; Everolimus; and combinations thereof. The agent can comprise paclitaxel. A mass of at least 200 μg of agent can be delivered to the venous wall. A mass of between 300 μg and 600 μg can be delivered to the venous wall. The method can further comprise implanting an anastomotic clip in the flow pathway and the agent can comprise a coating of the anastomotic clip. The agent can be applied to the vein wall by a delivery catheter. The agent can comprise a coating on a balloon of the delivery catheter. The delivery catheter can comprise a permeable balloon and the agent can be delivered through the permeable balloon.

In some embodiments, the method further comprises implanting a venous stent in a vein proximate the flow pathway. The implanting a venous stent can comprise implanting multiple venous stents. The implanting a venous stent can be configured to treat an existing venous stenosis. The flow pathway can be configured to create an elevated flow condition in the stented portion of the vein. The existing venous stenosis can comprise a deep vein thrombosis. The method can further comprise occluding the flow pathway. The flow pathway can be occluded by implanting a covered stent in the flow pathway. The flow pathway can be occluded at least one week after the implantation of the venous stent. The flow pathway can be occluded after a duration of time selected from the group consisting of: 1 week; 1 month; 3 months; and 6 months. The venous stent can be implanted to reduce future venous stenosis creation. The venous stent can comprise one or more venous stents comprising a diameter between 2 mm and 16 mm.

In some embodiments, the method further comprises implanting a covered stent in a vein to at least partially occlude the flow pathway.

According to another aspect of the technology, a system of treating angina in a patient comprises a needle delivery device constructed and arranged to place a vessel-to-vessel guidewire from a starting vessel to a target vessel and a flow creation device constructed and arranged to be advanced over the vessel-to-vessel guidewire and to create a flow pathway between the starting vessel and the target vessel. The system is constructed and arranged to at least reduce the angina of the patient.

According to another aspect of the technology, a system of treating mitral regurgitation in a patient comprises a needle delivery device constructed and arranged to place a vessel-to-vessel guidewire from a starting vessel to a target vessel, and a flow creation device constructed and arranged to be advanced over the vessel-to-vessel guidewire and to create a flow pathway between the starting vessel and the target vessel. The system is constructed and arranged to at least reduce the mitral regurgitation of the patient.

In some embodiments, the needle delivery device comprises an advanceable needle.

In some embodiments, the needle delivery device comprises a needle with a gauge between 20 and 24. The needle can comprise an approximately 22 gauge needle.

In some embodiments, the needle delivery device comprises a curved needle. The needle delivery device can further comprise a marker indicating the direction of curvature of the curved needle. The marker can comprise a marker selected from the group consisting of: flat surface, visible marker, line, textured surface, and combinations thereof. The needle delivery device can further comprise a sheath constructed and arranged to slidingly receive the curved needle. The needle can comprise a proximal end and a hub positioned on the proximal end. The hub can be constructed and arranged to be advanced to advance the curved needle out of the sheath.

In some embodiments, the needle delivery device comprises a needle comprising a shaped memory alloy. The shaped memory alloy can comprise nickel titanium alloy.

In some embodiments, the system further comprises a vessel-to-vessel guidewire constructed and arranged to be placed from the starting vessel to the target vessel by the needle delivery device. The vessel-to-vessel guidewire can comprise a wire with an outer diameter approximating 0.018″. The vessel-to-vessel guidewire can comprise a marker. The marker can be positioned to indicate the flow pathway location. The vessel-to-vessel guidewire can comprise a distal portion and a mid-portion and the mid portion can comprise a construction different than the construction of the distal portion. The mid portion can comprise a stiffness greater than the stiffness of the distal portion.

In some embodiments, the flow creation device comprises a balloon catheter configured to dilate tissue positioned between the starting vessel and the target vessel.

In some embodiments, the flow creation device comprises an energy delivery device constructed and arranged to deliver energy to tissue positioned between the starting vessel and the target vessel.

In some embodiments, the flow creation device comprises a clip deployment catheter comprising an anastomotic clip. The clip deployment catheter can comprise a handle and the handle comprises a control constructed and arranged to deploy the anastomotic clip. The control can comprise a button. The handle can comprise a safety position for the control. The handle can comprise a longitudinal axis and the control can be constructed and arranged to be moved relatively perpendicular to the longitudinal axis to transition from the safety position to a first ready to deploy position. The clip can comprise at least two distal arms, and the handle can be constructed and arranged to allow an operator to move the control from a first ready to deploy position to a first deployed position, and the movement causes the at least two distal arms to be deployed. The handle can comprise a longitudinal axis and the control can be moved relatively parallel to the longitudinal axis to transition from the first ready to deploy position to the first deployed position. The handle can be constructed and arranged to allow an operator to move the control from the first deployed position to a second ready to deploy position. The control can be moved relatively perpendicular to the longitudinal axis to transition from the first deployed position to the second ready to deploy position. The clip can comprise at least two proximal arms, and the handle can be constructed and arranged to allow an operator to move the control from the second ready to deploy position to a second deployed position, and the movement causes the at least two proximal arms to be deployed. The control can be moved relatively parallel to the longitudinal axis to transition from the second ready to deploy position to the second deployed position. The clip deployment catheter can comprise an outer sheath and the control can be constructed and arranged to be moved from a first position to a second position to cause movement of the outer sheath. The clip deployment catheter can be constructed and arranged such that movement of the control to the second position causes a tactile feedback event to occur. The clip can comprise multiple deployable arms, and the clip deployment catheter can be constructed and arranged such that movement of the control to the second position causes at least one arm to be deployed. At least one of the clip deployment catheter or the clip can comprise at least one marker constructed and arranged to rotationally position the clip. The marker can be constructed and arranged to be oriented toward the target vessel prior to deployment of the clip. The marker can be oriented based on a patient image. The patient image can comprise a real-time fluoroscopy image. The clip can comprise a swing arm for deployment in the target vessel and the marker can be positioned in alignment with the swing arm. The marker can be positioned on the clip. The clip deployment catheter can comprise a distal portion and the distal portion can comprise the clip and the marker. The marker can be positioned proximate the clip. The clip deployment catheter can comprise a proximal portion and the proximal portion can comprise the marker. The clip deployment catheter can comprise a handle and the marker can be positioned on the handle. At least one of the clip deployment catheter or the clip can comprise at least one marker constructed and arranged to longitudinally position the clip at the flow pathway location. The marker can indicate the distal end of the clip. The marker can indicate the proximal end of the clip. The clip can comprise multiple deployable arms, and the clip deployment catheter can be constructed and arranged to deploy at least one of the deployable arms and subsequently recapture one of the deployable arms. The clip deployment catheter can be constructed and arranged to be rotated and simultaneously deployed from the starting vessel to the target vessel over the vessel-to-vessel guidewire. The clip deployment catheter can comprise a projection constructed and arranged to mechanically engage the clip. The projection can comprise a pin. The clip deployment catheter can further comprise a second projection constructed and arranged to mechanically engage the clip.

In some embodiments, the system further comprises a flow pathway maintaining implant. The flow pathway maintaining implant can comprise an anastomotic clip. The clip can comprise a plurality of distal arms and a plurality of proximal arms and the distal arms can be independently deployable from the proximal arms. The clip can comprise four deployable distal arms. The clip can comprise four deployable proximal arms. The clip can comprise nickel titanium alloy. The clip can comprise multiple deployable arms and at least two arms can comprise a marker. The marker can comprise a radiopaque marker. The flow pathway maintaining implant can comprise suture. The flow pathway maintaining implant can comprise one or more staples. The flow pathway maintaining implant can comprise adhesive. The flow pathway maintaining implant can comprise at least a portion that comprises biodegradable material. The flow pathway maintaining implant can comprise a covered portion. The covered portion can be constructed and arranged to direct the flow of blood to and/or from the flow pathway. The covered portion can be constructed and arranged to limit flow of blood to a single direction in the target vessel and/or starting vessel. The covered portion can be constructed and arranged to reduce a future venous stenosis. The covered portion can comprise a covering material selected from the group consisting of: PTFE; nickel titanium alloy; polyurethane; one or more bioerodible polymers; a woven mesh of polymers; a woven mesh of nickel titanium fibers; and combinations thereof.

In some embodiments, the system further comprises a venous system introducer. The venous system introducer can be constructed and arranged to access the starting vessel. The venous system introducer can comprise an 11 French introducer. The venous system introducer can comprise a beveled distal tip. The beveled distal tip can comprise an angle between 20° and 50°. The beveled distal tip can comprise an angle of approximately 30°. The venous system introducer can comprise a marker proximate the beveled distal tip. The marker can comprise a radiopaque marker. The venous system introducer can comprise a proximal portion comprising a marker, and the marker can be aligned with the beveled distal tip. The venous system introducer can comprise a distal portion and an expandable element mounted to the distal portion. The expandable element can comprise a balloon. The expandable element can be constructed and arranged to prevent inadvertent advancement of the introducer into the target vessel. The venous system introducer can be constructed and arranged to stabilize the starting vessel.

In some embodiments, the system further comprises an arterial system introducer. The arterial system introducer can be constructed and arranged to access the target vessel. The arterial system introducer can comprise a 4 French introducer.

In some embodiments, the system further comprises a target wire constructed and arranged for positioning in the target vessel. The target wire can comprise a helical distal portion. The target wire can comprise a radiopaque distal portion.

In some embodiments, the system further comprises a flow pathway modifying device. The flow pathway modifying device can comprise an expandable element. The expandable element can be constructed and arranged to expand to a diameter between 3 mm and 5 mm. The expandable element can be constructed and arranged to expand to a diameter of approximately 4 mm. The expandable element can comprise a balloon. The expandable element can comprise at least one of an expandable cage or radially deployable arms. The flow modifying device can comprise a device selected from the group consisting of: an over the wire device constructed and arranged to be delivered over a vessel-to-vessel guidewire as described herein; an expanding scaffold configured to increase or otherwise modify flow pathway geometry such as an expandable balloon; an energy delivery catheter such as a catheter configured to deliver energy to tissue proximate a flow pathway; an agent delivery catheter such as a catheter configured to deliver an agent such as a pharmaceutical agent or an adhesive such as fibrin glue; and combinations thereof.

In some embodiments, the system further comprises a patient imaging apparatus. The patient imaging apparatus can comprise a fluoroscope. The patient imaging apparatus can comprise an ultrasound imager.

In some embodiments, the system is constructed and arranged to treat a patient disease or disorder selected from the group consisting of: angina; angina pectoris; mitral regurgitation; venous stenosis; deep vein thrombosis; hypertension; arterial hypertension; chronic obstructive pulmonary disease; congestive heart failure; lung fibrosis; adult respiratory distress syndrome; lymphangioleiomyomatosis; pulmonary hypertension; sleep apnea such as sleep apnea due to hypoxemia or hypertension; and combinations thereof.

In some embodiments, the system further comprises a venous stent. The venous stent can comprise a self-expanding stent. The venous stent can comprise a balloon expandable stent. The venous stent can comprise multiple venous stents. The multiple venous stents can comprise a first stent with a different parameter than a second stent, and the different parameter can comprise a parameter selected from the group consisting of: length; cutaway dimension; cutaway location relative to an end of the venous stent; diameter; pattern; stent mesh pattern; number of radiopaque markers; position of a radiopaque marker; tensile strength; hoop strength; and combinations thereof. The stent can be constructed and arranged to treat a venous stenosis. The venous stenosis can comprise a deep vein thrombosis. The venous stent can be constructed and arranged to be implanted in a vein at a location at least one of: over, adjacent or proximate the flow pathway. The venous stent can be constructed and arranged to reduce a future venous stenosis. The venous stent can comprise one or more stents comprising a diameter between 2 mm and 16 mm. The venous stent can comprise at least one venous stent comprising a cutaway end portion. The venous stent can comprise at least one venous stent comprising an opening positioned along the length of the venous stent. The opening can comprise a rectangular shaped opening. The opening can comprise an oval shaped opening. The venous stent can comprise at least one venous stent comprising a c-shaped profile. The venous stent can comprise at least one venous stent comprising a c-shaped end portion. The system can further comprise a delivery sheath configured to implant the venous stent. The venous stent can comprise an opening along its length, and a marker constructed and arranged to identify the opening. The system can further comprise an anastomotic clip constructed and arranged to mechanically engage the venous stent.

In some embodiments, the system further comprises a covered stent. The covered stent can be constructed and arranged to be implanted to at least partially occlude the flow pathway. The system can further comprise a venous stent configured to treat a venous stenosis, and the flow pathway can be created to elevate flow within the venous stent, and the covered stent can be constructed and arranged to occlude the flow pathway at least one week after the creation of the flow pathway. The covered stent can comprise a PTFE covered stent. The system can further comprise a catheter constructed and arranged to implant the covered stent.

In some embodiments, the system further comprises an agent configured to reduce a venous stenosis. The agent can be configured to prevent the creation of a future venous stenosis proximate the flow pathway. The agent can comprise an agent selected from the group consisting of: anti-proliferative agent; a chemotherapeutic agent; paclitaxel; an mTOR inhibitor; Sirolimus; Zotarolimus; Everolimus; and combinations thereof. The agent can comprise paclitaxel. The system can further comprise a catheter configured to deliver the agent to tissue.

In some embodiments, the system further comprises an agent delivery catheter configured to deliver an agent to a vessel wall. The agent delivery catheter can be constructed and arranged to reduce a future vessel stenosis. The agent delivery catheter can comprise a balloon. The agent delivery catheter can further comprise a coating on the balloon comprising the agent to be delivered to the vessel wall. The balloon can comprise a permeable balloon constructed and arranged to allow agent to pass therethrough and into the vessel wall. The system can further comprise an agent to be delivered to the vessel wall by the agent delivery catheter. The agent can comprise an agent selected from the group consisting of: anti-proliferative agent; a chemotherapeutic agent; paclitaxel; an mTOR inhibitor; Sirolimus; Zotarolimus; Everolimus; and combinations thereof. The agent can comprise paclitaxel.

According to another aspect of the technology, a method of treating venous stenosis of a patient comprises selecting a patient exhibiting a venous stenosis, implanting a stent in the stenotic vein proximate the stenosis, and creating a flow pathway between a first vascular location and a second vascular location. The flow pathway is positioned proximate the implanted stent and the first vascular location comprises the stenotic vein.

In some embodiments, the second vascular location comprises a source of arterial blood. The source of arterial blood can comprise an artery. The artery can comprise an artery proximate the stenotic vein.

In some embodiments, the flow pathway is configured to create an elevated flow condition in the stented portion of the stenotic vein.

In some embodiments, the method further comprises occluding the flow pathway. The flow pathway can be occluded at least one week after creation of the flow pathway. The flow pathway can be occluded after a minimum time period after the creation of the flow pathway, the minimum time period selected from the group consisting of: 1 week; 1 month; 3 months; or 6 months. Occluding the flow pathway can comprise implanting a covered stent over the flow pathway.

In some embodiments, the stenotic vein comprises a stenotic femoral vein.

### BRIEF DESCRIPTION OF THE DRAWINGS

The accompanying drawings, which are incorporated in and constitute a part of this specification, illustrate various embodiments of the present inventive concepts, and, together with the description, serve to explain the principles of the invention. In the drawings:

FIG. 1 is a flow chart of a method for treating a patient by creating a flow pathway between a first vascular location and a second vascular location, consistent with the present inventive concepts.

FIG. 2 is a schematic view of a system for creating a flow pathway in a patient, consistent with the present inventive concepts.

FIGS. 3A through 3D are a set of steps for implanting an anastomotic clip, consistent with the present inventive concepts.

FIG. 4 is a flow chart of a method for treating a patient with a flow pathway, consistent with the present inventive concepts.

FIG. 5 is an angiographic view of a patient's vein and artery prior to advancement of a needle into the artery, consistent with the present inventive concepts.

FIGS. 5A, 5B and 5C are anatomical views of three different needle trajectory paths, consistent with the present inventive concepts.

FIG. 6 is a perspective view of an anastomotic clip, consistent with the present inventive concepts.

FIG. 7 is a side and end view of a venous stent including a cutaway end, consistent with the present inventive concepts.

FIG. 8 is a side and end view of a venous stent including an opening along its length, consistent with the present inventive concepts.

FIG. 9 is a side and end view of a venous stent including a c-shaped profile, consistent with the present inventive concepts.

FIG. 10 is a side and end view of a venous stent including a c-shaped end portion, consistent with the present inventive concepts.

FIG. 11 is a side view of a venous stent being deployed from a delivery sheath, consistent with the present inventive concepts.

FIG. 12 is a side sectional, anatomical view of an anastomotic clip and a venous stent positioned in and near to but away from, respectively, a flow pathway, consistent with the present inventive concepts.

FIG. 13 is a side sectional, anatomical view of an anastomotic clip, including a covered portion, positioned in a flow pathway, consistent with the present inventive concepts.

FIG. 13A is a side sectional, anatomical view of the flow pathway of FIG. 13, showing only the covered portion of the anastomotic clip, consistent with the present inventive concepts.

FIG. 14 is a flow chart of a method for treating a venous stenosis, consistent with the present inventive concepts.

FIG. 15 is a side sectional, anatomical view of a system including a venous stent positioned at a venous stenosis and an anastomotic clip positioned in a flow pathway, consistent with the present inventive concepts.

FIG. 16 is a side sectional, anatomical view of the system of FIG. 15, further including a covered stent positioned in the vein to occlude the flow pathway, consistent with the present inventive concepts.

### DETAILED DESCRIPTION OF THE DRAWINGS

Reference will now be made in detail to the present embodiments of the inventive concepts, examples of which are illustrated in the accompanying drawings. Wherever practical, the same reference numbers will be used throughout the drawings to refer to the same or like parts.

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on” or “connected” or “coupled” to another element, it can be directly on or above, or connected or coupled to, the other element or intervening elements can be present. In contrast, when an element is referred to as being “directly on” or “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.). When an element is referred to herein as being “over” another element, it can be over or under the other element, and either directly coupled to the other element, or intervening elements may be present, or the elements may be spaced apart by a void or gap.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The systems, devices and methods of the present inventive concepts include creating a flow pathway between a first vascular location (e.g. a source of arterial blood) and a second vascular location (e.g. a source of venous blood). In some embodiments, multiple flow pathways are created. The one or more flow pathways can be configured to treat one or more diseases or disorders, such as angina. In some embodiments, the one or more flow pathways are configured to treat mitral regurgitation (i.e. mitral valve regurgitation). In some embodiments, the one or more flow pathways are created to improve the long term patency of one or more stents placed to treat deep vein thrombosis (DVT) or other venous stenosis (i.e. narrowing of the vein). In these embodiments, flow is increased through the one or more stents by a flow pathway created between the stented vein and a neighboring artery. In a subsequent procedure (e.g. a procedure performed at least 1 week, at least 1 month, at least 3 months or at least 6 months after the flow pathway creation procedure), the flow pathway can be fully or at least partially occluded, such as by placing a covered stent (e.g. a PTFE covered stent) in the artery to cover at least a portion of an end of the flow pathway (at least partially cover an end of the flow pathway at the venous wall or the arterial wall). In some embodiments, the one or more flow pathways are configured to treat a patient disease or disorder selected from the group consisting of: angina; angina pectoris, mitral regurgitation; venous stenosis; deep vein thrombosis; hypertension; arterial hypertension; chronic obstructive pulmonary disease (COPD); congestive heart failure; lung fibrosis; adult respiratory distress syndrome; lymphangioleiomyomatosis; pulmonary hypertension; sleep apnea such as sleep apnea due to hypoxemia or hypertension; and combinations of one or more of these.

The systems and devices of the present inventive concepts can include one or more anastomotic clips constructed and arranged to be placed in a flow pathway between a first vascular location (e.g. a source of arterial blood) and a second vascular location (e.g. a source of venous blood). In some embodiments, the anastomotic clip includes a covered portion. The covered portion can be constructed and arranged to direct the flow of blood to and/or from the flow pathway, such as to limit flow of blood from the flow pathway to a single direction within a receiving vessel (e.g. a vein) and/or to prevent or at least reduce (hereinafter reduce) a current (already existing) or future vessel stenosis (e.g. a venous stenosis).

The systems and devices of the present inventive concepts can include a venous stent configured to be positioned over, adjacent to and/or otherwise proximate a flow pathway between a first vascular location (e.g. a source of arterial blood) and a second vascular location (e.g. a source of venous blood). The venous stent can be constructed and arranged to reduce a current and/or future venous stenosis. The venous stent can include a cutaway portion or other opening configured to be positioned relatively aligned (axially and rotationally) with the opening in the vein wall at the venous wall end of the flow pathway (hereinafter “positioned over the flow pathway”).

The systems and devices of the present inventive concepts can include an agent and/or an agent-delivering device (e.g. an agent delivery catheter) configured to treat tissue within or otherwise proximate a flow pathway between a first vascular location (e.g. a source of arterial blood) and a second vascular location (e.g. a source of venous blood). The agent-delivering device can comprise an agent-delivering balloon that is configured to treat tissue to reduce a current and/or future vessel stenosis, such as a venous stenosis. The agent-delivering balloon can comprise a balloon coated with one or more stenosis reducing drugs or other agents and/or a permeable balloon configured to deliver the one or more agents.

The systems and devices of the present inventive concepts can include a covered stent used to at least partially occlude a flow pathway, such as a flow pathway of the present inventive concepts. The covered stent can be placed in a vein or artery, such that a covered portion of the stent covers at least a portion of the flow pathway, such as to fully occlude the flow pathway at least twenty-four hours after the creation of the flow pathway.

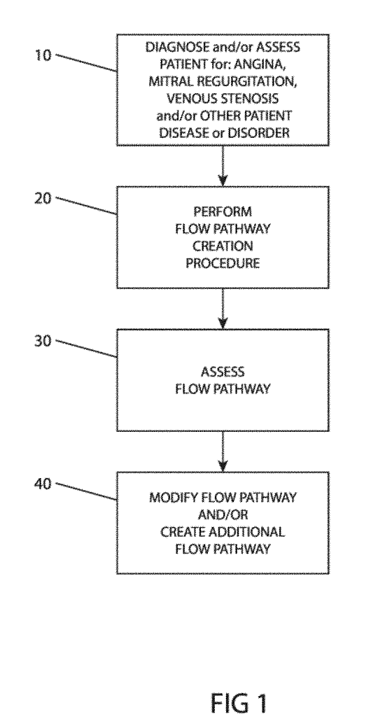

Referring now to FIG. 1, a flow chart for selecting and treating a patient by creating at least one fistula or other flow pathway between a first vascular location in the patient's arterial system and a second vascular location in the patient's venous system is illustrated, consistent with the present inventive concepts. In STEP 10, a patient assessment is performed, such as to diagnose the patient and determine whether one or more flow pathways (hereinafter “flow pathway” for either a single flow pathway or multiple flow pathways) should be created in the patient. A patient can be selected based on a disease or disorder which is diagnosed in STEP 10 or previously. In some embodiments, a patient diagnosed with angina and/or mitral regurgitation is selected to receive a flow pathway. The flow pathway can be created to eliminate or at least decrease angina and/or mitral regurgitation in a patient. The patient can exhibit one or more forms of angina, such as angina pectoris. The patient can exhibit one or more forms of mitral regurgitation, such as: Type I mitral regurgitation; Type II mitral regurgitation; Type III mitral regurgitation; mild mitral regurgitation; moderate mitral regurgitation; moderate to severe mitral regurgitation; severe mitral regurgitation; and combinations of one or more of these. Alternatively or additionally, a patient can be selected to receive a flow pathway to treat a disease or disorder selected from the group consisting of: hypertension; arterial hypertension; chronic obstructive pulmonary disease (COPD); congestive heart failure; lung fibrosis; adult respiratory distress syndrome; lymphangioleiomyomatosis; pulmonary hypertension; sleep apnea such as sleep apnea due to hypoxemia or hypertension; arrhythmia; erectile dysfunction; orthostatic intolerance; and combinations of one or more of these. The flow pathway can be created to reduce arterial hypertension of the patient. The flow pathway can be created to increase cardiac output of the patient. The flow pathway can be created to increase the flow of blood to the right side of the heart, such as to increase cardiac output of the patient. The flow pathway can be created to cause a physiologic change in the patient selected from the group consisting of: increased oxygen delivery by the arterial system; increased blood volume; increased proportion of blood flow to the descending aorta; increased blood flow to the kidneys; increased blood flow outside the kidneys; increased cardiac output; and combinations of one or more of these.

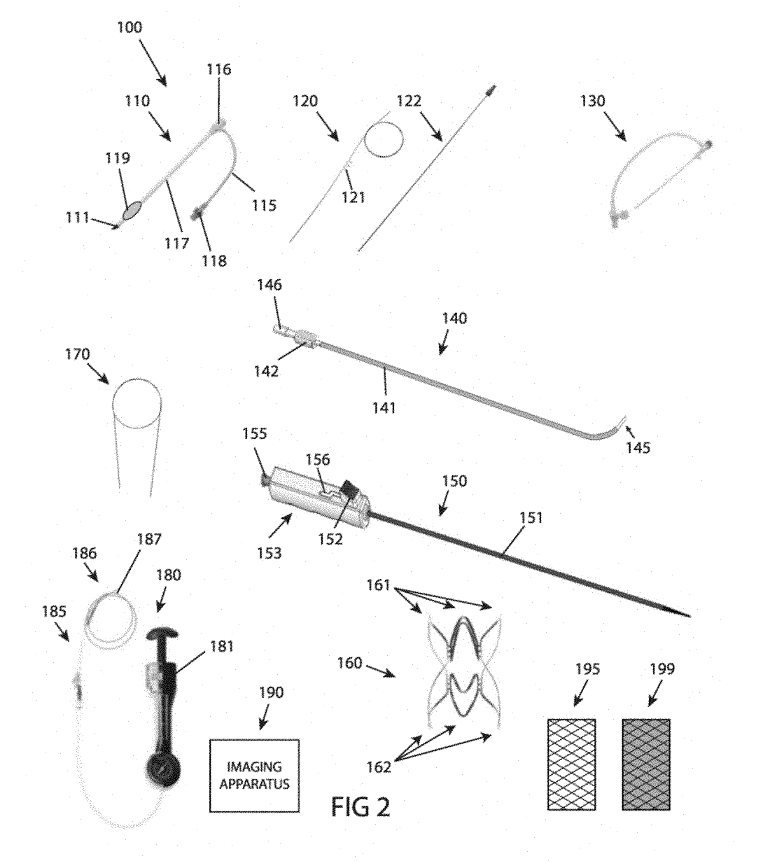

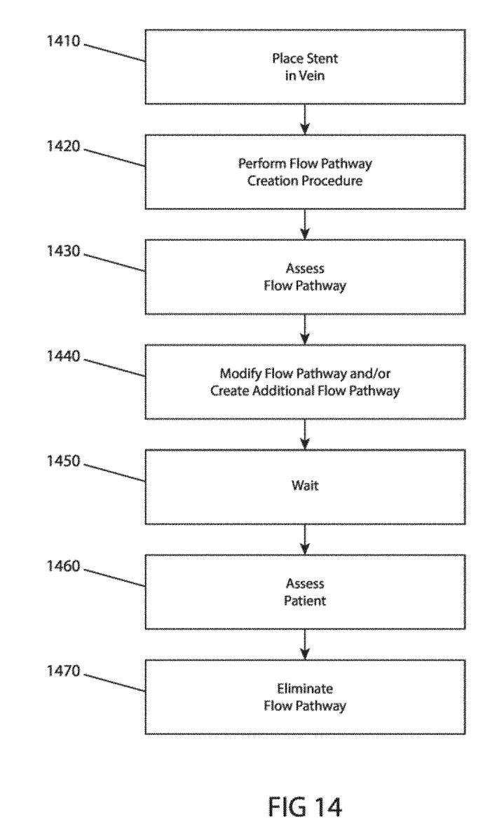

In some embodiments, creation of a flow pathway is determined to be included as part of a venous stenting procedure, such as a procedure in which one or more stents are placed in a vein (e.g. to treat deep vein thrombosis or other venous stenosis) and the flow pathway is created to increase the flow through the one or more stents (e.g. two to four stents placed in the vein) to prevent venous collapse or otherwise improve long-term patency of the associated vein (e.g. as described herebelow in reference to FIGS. 14-16). The created flow pathway can be subsequently occluded (e.g. in an additional procedure performed at least 1 week, at least 1 month, at least 3 months or at least 6 months after the flow pathway creation procedure), such as by placing a covered stent (e.g. covered stent 199 described herebelow in reference to FIG. 2) in the artery to at least partially occlude the flow pathway, such as a flow pathway occlusion procedure performed in STEP 40 described herebelow. In some embodiments, at least one venous stent is placed in the ilio-femoral veins to treat deep vein thrombosis (DVT), and a flow pathway is created to increase flow through the at least one stent.

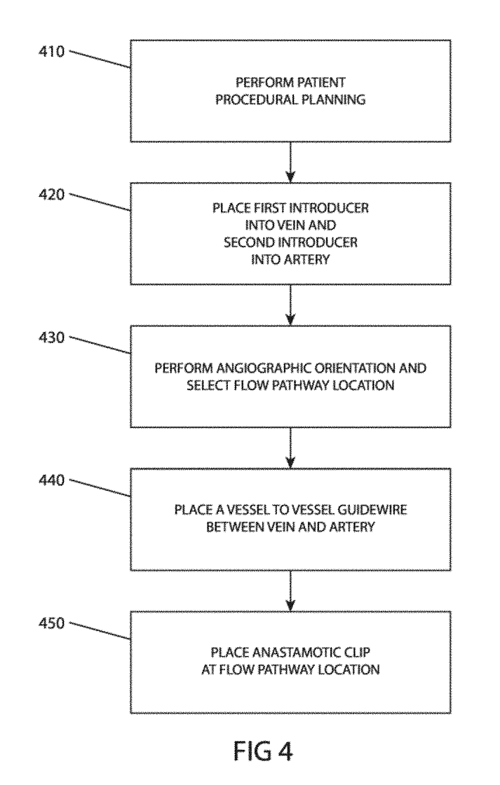

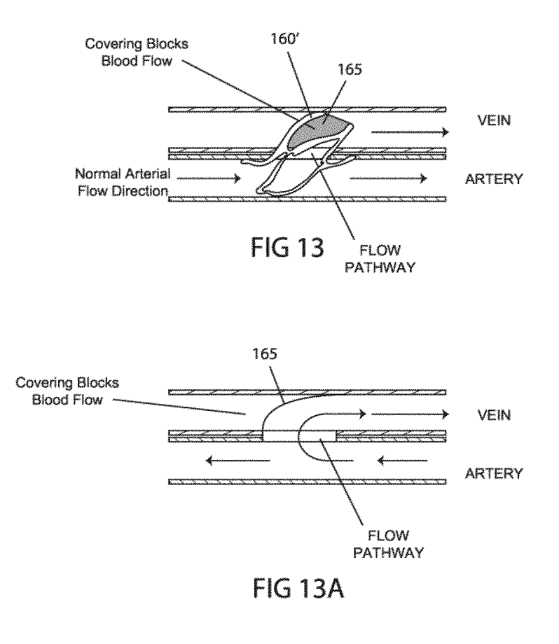

In STEP 20, a flow pathway creation procedure is performed on the patient. In some embodiments, the flow pathway creation procedure is performed as described herebelow in reference to FIG. 4. In some embodiments, the flow pathway creation procedure is performed using a system of devices and components similar to system 100 described herebelow in reference to FIG. 2. The flow pathway is created between a first vascular location in the arterial system, such as an artery, and a second vascular location in the venous system, such as a vein. The flow pathway creation procedure can comprise a clinical procedure selected from the group consisting of: a surgical procedure such as a surgical procedure performed in an operating room; an interventional procedure such as an interventional procedure performed in a catheterization lab or radiology lab; and combinations of one or more of these. The flow pathway creation procedure can include the placement of a vessel-to-vessel guidewire between a starting vessel such as a vein (e.g. an iliac vein), and a target vessel such as an artery (e.g. an iliac artery). In these embodiments, the flow pathway can be created using one or more flow pathway creation devices that are advanced over the vessel-to-vessel guidewire. An anastomotic clip or other implant (e.g. anastomotic clip 160 or 160′ described herebelow) can be placed into the flow pathway via a clip placement device advanced over the vessel-to-vessel guidewire. Alternatively, a flow pathway can be created without an anastomotic clip, such as through placement of suture and/or staples (e.g. in an open surgery or via an over-the-wire suture and/or staple delivery device). One or more implants placed within and/or otherwise proximate the flow pathway can be biodegradable or comprise one or more biodegradable portions. In some embodiments, a tissue treatment procedure can be performed to create the flow pathway (e.g. with or without an implant) such as a procedure using energy (e.g. radiofrequency energy) or an adhesive (e.g. fibrin glue) coating applied to the tissue surrounding or otherwise proximate the flow pathway. One or more flow pathway treatment or modification procedures can be performed using flow pathway treatment or modification devices advanced over the vessel-to-vessel guidewire, such as the flow pathway modification procedure performed in STEP 40 herebelow. In some embodiments, the anastomotic clip comprises a covered portion, such as is described herebelow in reference to clip 160′ of FIG. 13. The covered portion can be constructed and arranged to direct the flow of blood to and/or from the flow pathway, such as to limit the flow of blood from the flow pathway to a single direction within a receiving vessel (e.g. a vein) and/or to reduce an existing and/or future vessel stenosis (e.g. a venous stenosis).

In some embodiments, at least one fistula or other flow pathway is created between an artery and a vein at a location distal to the renal arteries (i.e. at an infrarenal location such as an infrarenal flow pathway created between an iliac artery and an iliac vein). In some embodiments, a flow pathway is created proximate a kidney. Numerous locations for the fistula or other flow pathway can be selected, such as a flow pathway located between an artery and vein as described herebelow in reference to FIG. 4. Alternatively or additionally, a flow pathway can be created between a chamber of the heart and a second vascular location, such as between the left atrium and the right atrium or between the left ventricle and the heart's coronary sinus. Alternatively or additionally, arterial blood can be diverted to the venous system by way of a flow pathway comprising a bypass graft, such as is described in applicant's U.S. Pat. No. 8,016,782, entitled “Methods for Providing Oxygenated Blood to Venous Circulation”, filed Jun. 13, 2005, the contents of which are incorporated by reference herein in its entirety.

During the flow pathway creation procedure of STEP 20 and/or in a subsequent flow pathway modification procedure (e.g. STEP 40), a flow pathway dilation procedure can be performed in which tissue within and/or otherwise proximate the flow pathway is dilated, such as with a balloon catheter. In some embodiments, an anastomotic clip is placed in the flow pathway and a balloon catheter is used to dilate the flow pathway and anastomotic clip simultaneously. In some embodiments, the dilating balloon comprises a diameter of approximately 3 mm to 6 mm, such as a diameter of approximately 4 mm or a diameter of approximately 5 mm In some embodiments, a single flow pathway is created to treat the patient, and the flow pathway flow rate comprises a flow rate of at least 400 ml/min and/or no more than 1500 ml/min, such as a flow rate of at least 600 ml/min and/or no more than 1000 ml/min In other embodiments, multiple flow pathways are created to treat the patient (e.g. in one or more clinical procedures), and the cumulative flow rate through the multiple flow pathways comprises a flow rate of at least 400 ml/min and/or no more than 1500 ml/min.

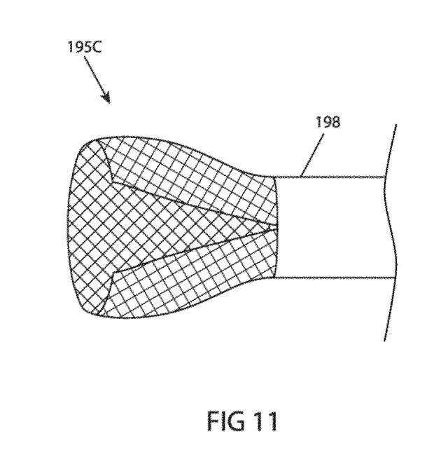

In some embodiments, during the flow pathway creation procedure of STEP 20 and/or in a subsequent flow pathway modification procedure (e.g. STEP 40), one or more venous stents are placed in the vein, such as at a location over, adjacent and/or otherwise proximate the flow pathway. The one or more venous stents can be of similar construction and arrangement to venous stent 195, 195a, 195b, 195c or 195d described herebelow in reference to FIG. 2, 7, 8, 9 or 10, respectively. The one or more venous stents can be placed via a delivery catheter, such as delivery sheath 198 described herebelow in reference to FIG. 11.

In some embodiments, during the flow pathway creation procedure of STEP 20 and/or in a subsequent flow pathway modification procedure (e.g. STEP 40), an agent-delivering device is positioned to deliver one or more agents to tissue, such as vessel wall tissue (e.g. venous wall tissue) to reduce a current and/or future vessel stenosis (e.g. venous stenosis). The agent-delivering device can be of similar construction and arrangement to balloon catheter 185 described herebelow in reference to FIG. 2.

In STEP 30, a flow pathway assessment procedure can be performed. STEP 30 can be performed in the same clinical procedure as STEP 20, and/or in a subsequent clinical procedure such as a procedure at least twenty-four hours after completion of STEP 20, or at least 1 week, at least 1 month, and/or at least 6 months after completion of STEP 20. In some embodiments, the assessment performed in STEP 30 includes one or more anatomical measurements, such as a measurement selected from the group consisting of: a flow pathway diameter measurement; a flow pathway length measurement; a measurement of the distance between an artery and vein comprising the flow pathway; a measurement of the distance between the flow pathway and a vessel sidebranch; and combinations of one or more of these. In some embodiments, the assessment performed in STEP 30 comprises an assessment of flow (e.g. flow within and/or otherwise proximate the flow pathway), such as a flow assessment selected from the group consisting of: flow through the flow pathway; flow in a vessel segment proximate the flow pathway; flow measured using Doppler ultrasound; flow measured using angiographic techniques; and combinations of one or more of these. In some embodiments, the assessment performed in STEP 30 comprises an assessment of a patient physiologic condition, such as an assessment of a physiologic condition selected from the group consisting of: angina level; mitral regurgitation type; mitral regurgitation severity; orthostatic intolerance level; dizziness level; lightheadedness level; syncope events; nausea level; fatigue level; tremor state; breathing state; swallowing ability; headache level; visual disturbance level; sweating level; pallor state; cardiac output; blood pressure such as systolic and/or diastolic blood pressure; respiration; a blood gas parameter; blood flow such as blood flow through a vein or artery within and/or otherwise proximate the right side of the heart; vascular resistance; pulmonary resistance; average clotting time assessment; serum creatinine level assessment; and combinations of one or more of these.

In STEP 40, one or more flow pathway parameters can be modified. STEP 40 can be performed in the same clinical procedure as STEP 20, and/or in a subsequent clinical procedure such as a procedure at least twenty-four hours after completion of STEP 20, or at least 1 week, at least 1 month, and/or at least 6 months after completion of STEP 20. In some embodiments, STEP 30 and STEP 40 are performed in the same clinical procedure (e.g. both in the same clinical procedure as STEP 20 or both in a subsequent clinical procedure). In some embodiments, one or more patient and/or flow pathway parameters to be modified are selected from the group consisting of: flow pathway cross sectional diameter; flow pathway average cross sectional diameter; flow pathway flow rate; flow pathway average flow rate; diastolic pressure after flow pathway creation; diastolic pressure change after flow pathway creation (e.g. as compared to diastolic pressure prior to flow pathway creation); systolic pressure after flow pathway creation; systolic pressure change after flow pathway creation (e.g. as compared to systolic pressure prior to flow pathway creation); ratio of diastolic to systolic pressure after flow pathway creation; difference between diastolic pressure and systolic pressure after flow pathway creation; and combinations of one or more of these.

Flow pathway modification procedures can include but are not limited to: increasing flow through the flow pathway; decreasing flow through the flow pathway; increasing the diameter of at least a segment of the flow pathway; decreasing the diameter of at least a segment of the flow pathway; removing tissue proximate the flow pathway; stenting a stenosis proximate the flow pathway (e.g. stenting a venous stenosis); delivering an agent to the flow pathway; delivering an agent to tissue proximate the flow pathway (e.g. venous and/or arterial wall tissue proximate the flow pathway); blocking a sidebranch proximate the flow pathway; and combinations of one or more of these. A flow pathway modifying device can include one or more devices selected from the group consisting of: an over the wire device constructed and arranged to be delivered over a vessel-to-vessel guidewire as described herein; an expanding scaffold configured to increase or otherwise modify flow pathway geometry such as an expandable balloon; a stent configured to be placed in an artery or vein proximate the flow pathway; an energy delivery catheter such as a catheter configured to deliver energy to tissue proximate a flow pathway; an agent delivery catheter such as a catheter configured to deliver an agent such as a pharmaceutical agent (e.g. a venous stenosis treating agent as described herebelow) or an adhesive such as fibrin glue; and combinations of one or more of these.

In some embodiments, the flow pathway comprises a diameter of at least 2.5 mm, such as a diameter of at least 3.0 mm In some embodiments, the flow pathway comprises a diameter of no more than 6.0 mm, such as a diameter of no more than 5.0 mm or 4.0 mm In some embodiments, the flow pathway comprises a diameter and/or a target flow rate that is based on a patient parameter, such as a measured patient parameter. The patient parameter can be determined prior to the creation of the flow pathway, during creation of the flow pathway and/or after the creation of the flow pathway (e.g. when the flow pathway is modified based on the patient parameter). In these embodiments, the patient parameter on which the flow pathway diameter and/or target flow rate is based can comprise a parameter selected from the group consisting of: angina level; mitral regurgitation severity; mitral regurgitation type; cardiac output; blood pressure; flow rate; tilt table test result; ankle brachial index test result; venous insufficiency level; peripheral vascular resistance; shunt flow; pulmonary capillary wedge pressure; right atrial pressure; pulmonary pressure; left atrial pressure; arterial oxygenation; venous oxygenation; and combinations thereof.

In some embodiments, a second fistula or other second flow pathway is created, such as using the techniques of STEP 20 described hereabove. The second flow pathway can be created in the same clinical procedure as STEP 20 (in which the first flow pathway is created), or in a subsequent clinical procedure such as a procedure performed at least twenty-four hours after completion of STEP 20, or at least 1 week, at least 1 month, and/or at least 6 months after completion of STEP 20. A second flow pathway can be created due to inadequate therapy provided by the first flow pathway, and/or if the first flow pathway has insufficient flow (e.g. narrows or becomes non-patent). A second flow pathway can be created due to formation of a vascular (e.g. venous) stenosis proximate the first flow pathway. In these embodiments, the first flow pathway can be reversed (e.g. closed), such as through the placement of a covered stent (e.g. covered stent 199 described herebelow in reference to FIG. 2) in the vein or artery that covers the flow pathway, or other flow pathway-occlusive procedure. Alternatively or additionally, the venous stenosis can be stented, such as by implanting one or more venous stents 195 as described herein. In some embodiments, three or more flow pathways can be created. Multiple flow pathways can be created in similar or dissimilar arteries and/or veins. In some embodiments, single or multiple flow pathways are created that exhibit a cumulative flow rate of at least 400 ml/min and/or no more than 1500 ml/min, such as a flow rate of at least 600 ml/min and/or no more than 1000 ml/min In some embodiments, a first flow pathway is created between the iliac artery and iliac vein in the patient's right leg, and a second flow pathway is created between the iliac artery and iliac vein in the patient's left leg.

The method of FIG. 1 can be performed using real-time imaging, such as real-time imaging provided by a fluoroscope and/or an ultrasound imaging device.

The method of FIG. 1 can be performed to decrease peripheral vascular resistance, such as to decrease infrarenal vascular resistance (e.g. below the kidneys or in a manner to include the great vessels of the aorta and/or the inferior vena cava). Alternatively or additionally, the method can be performed to achieve a physiologic change selected from the group consisting of: reduced angina; reduced mitral regurgitation; increased oxygen delivery by the arterial system; increased blood volume; increased proportion of blood flow to the descending aorta; increased blood flow to the kidneys; increased blood flow outside the kidneys; increased cardiac output; and combinations of one or more of these. The method can be constructed and arranged to prevent any significant chronic increase in heart rate. Alternatively or additionally, the method can be constructed and arranged to prevent a decrease in cardiac function. Alternatively or additionally, the method can be constructed and arranged to avoid undesired adverse effects to the kidneys, such as by avoiding the adverse effects that can be encountered in a renal denervation procedure, such as stenosis, lost autonomic control and/or vessel intima damage.

In some embodiments, the method is performed to increase oxygenation and/or flow rates associated with the patient's chemo-receptors, such as to cause a therapeutic change to vascular resistance. In some embodiments, the method is performed to affect or otherwise modify the patient's central sympathetic tone. Modifications to central sympathetic tone can be performed to reduce systolic and/or diastolic blood pressure (e.g. mean systolic and/or mean diastolic blood pressure), and/or to treat other patient diseases and conditions such as angina, mitral regurgitation, diabetes, sleep apnea, or heart failure.

In some embodiments, the method of FIG. 1 is constructed and arranged to cause a reduction in diastolic blood pressure that is equal to or greater than a concurrent reduction in systolic blood pressure. In some embodiments, the method is constructed and arranged to reduce the diastolic pressure more than the systolic pressure by an amount of at least 2 mmHg, at least 4 mmHg or approximately 5 mmHg In some embodiments, the method is constructed and arranged to reduce the diastolic pressure by at least 5 mmHg, such as a reduction of at least 10 mmHg, at least 15 mmHg or approximately 18 mmHg In some embodiments, the method is constructed and arranged to reduce the systolic pressure by at least 5 mmHg, such as a reduction of at least 10 mmHg or approximately 13 mmHg In some embodiments, the method is constructed and arranged to cause a reduction in blood pressure to a level at or below 130/90 mmHg.

In some embodiments, the method of FIG. 1 is constructed and arranged to cause one or more of: a decrease in systemic vascular resistance; a decrease in blood pressure; an increase in cardiac output; an increase in right atrial pressure; an atrial natriuretic peptide (ANP) release; vasodilation; an increase in right atrial filling; a Bainbridge reflex; an increase in heart rate; peripheral sympatho-inhibition; activation of venous baroreceptors; activation of pulmonary arterial mechanoreceptors; an increase in venous oxygenation; an increase in pulmonary blood flow; an increase in arterial compliance; a decrease in a reflected pulse wave; a decrease in effective arterial volume; an increase in oxygen delivery to tissue; a decrease in chemoreceptor activity; a decrease in sympatho-excitation (e.g. due to cerebral and/or renal hypoperfusion); sodium and/or water retention; reduced renal ischemia; and combinations of one or more of these.

As described hereabove in reference to STEP 10, the method of FIG. 1 can be constructed and arranged to treat angina. The created flow pathway and/or an anastomotic clip positioned therein (e.g. anastomotic clip 160 or 160′ described herebelow), can be configured to reduce cardiac work; reduce arterial pressure; reduce left ventricular pre-load; shift blood volume toward the venous system; increase systemic oxygenation; increase venous oxygenation; increase delivery of oxygen to tissue; and combinations of one or more of these. Creation of the flow pathway can be constructed and arranged to reduce the need for repetitive persistent medications, such as glyceryl trinitrate (also referred to as nitroglycerin or GTN), while allowing more patient activity.

As described hereabove in reference to STEP 10, the method of FIG. 1 can be constructed and arranged to create an elevated flow condition in a stented portion of a vein, such as is also described herebelow in reference to FIG. 2, 14, 15 or 16. In these embodiments, the flow pathway modification of STEP 40 can include placing a covered stent or other occlusion device to at least partially occlude the flow pathway (e.g. covered stent 199 described herebelow in reference to FIG. 2), such as to subsequently prevent shunting of blood from the artery to the stented vein. The occlusion via the covered stent can be performed at least 1 week after the stenting of the vein, such as an occlusion procedure performed at least 1 month, at least 3 months or at least 6 months after the creation of the flow pathway between the vein receiving the one or more stents and a proximate artery.

Referring now to FIG. 2, a system for creating at least one fistula or other flow pathway between a first location in the patient's arterial system (e.g. an artery), and a second location in the patient's venous system (e.g. a vein), is illustrated. System 100 can comprise a vascular introducer, first introducer 110, configured to be placed into the patient to provide access to a starting vessel. System 100 can comprise another vascular introducer, second introducer 130, configured to provide access to a target vessel. In some embodiments, the starting vessel is a vein, and the target vessel is an artery. In other embodiments, the starting vessel is an artery and the target vessel is a vein. System 100 can include target wire 120 which can comprise helical section 121 and can be configured to be placed through the second introducer 130 and into the target vessel. Target wire 120 can be placed through an elongate tube, catheter 122. System 100 can comprise needle deployment device 140 which can be configured to deploy crossing needle 145 (shown in an advanced position in FIG. 2), from the starting vessel and into the target vessel. System 100 can include a vessel-to-vessel guidewire 170, which can be placed from the starting vessel to the target vessel via needle deployment device 140. System 100 can also include clip deployment catheter 150, which can be configured to deploy anastomotic clip 160. System 100 can include one or more flow pathway modifying devices, such as one or more dilation devices 180 which can include one or more balloon catheters 185, each attachable to indeflator 181 (e.g. a standard angioplasty balloon indeflator). Catheter 185 can comprise at least one balloon 186. Balloon 186 can comprise an agent-coated balloon, including agent 187, such as is described in detail herebelow. Alternatively or additionally, balloon 186 can comprise at least a portion that is permeable and configured to deliver agent 187, also as described in detail herebelow. System 100 can further comprise imaging apparatus 190, typically a fluoroscope and/or ultrasound imaging device used to image one or more devices or components of system 100, as well as the patient's anatomy, during the creation of an arteriovenous flow pathway.

First introducer 110 can be configured to be placed into the patient to provide access to a starting vessel (e.g. a vein of a patient). In some embodiments, introducer 110 comprises an 11 French vascular introducer. First introducer 110 can comprise beveled tip 111 with an angle ranging from 20° to 50°, such as at an angle of approximately 30°. Additionally, system 100 can include a kit comprising an additional introducer having a second angle providing the clinician, clinician's assistant, operator or other user (hereinafter “clinician”, “operator” or “user”) with more options as may be appropriate for a particular patient's anatomical geometry. In some embodiments, beveled tip 111 comprises a marker, for example, a radiopaque or other visualizable marker, such that the luminal wall of the starting vessel can be imaged (e.g. when tip 111 is pressed against the vessel wall). The proximal portion of introducer 110 can comprise a contour or marker, such as to be correlated with or otherwise indicate the alignment of the bevel of tip 111.

Introducer 110 can comprise shaft 117 which includes at least one thru lumen. Introducer 110 can also comprise port 116, typically a hemostasis valve, which can be fluidly connected to the lumen of shaft 117. A second port 118, typically a luer connector, can be connected to tubing 115 which in turn can be connected to port 116. Introducer 110 can further comprise a dilator, not shown but typically an 11 to 13 French dilator used to introduce and/or pre-dilate tissue receiving introducer 110. Introducer 110 can further comprise a radially expandable element, such as expandable element 119, such as a balloon or expandable cage located on the distal portion of introducer 110. In some embodiments, expandable element 119 can be configured to prevent or otherwise resist advancement of introducer 110 into the target vessel. Alternatively or additionally, expandable element 119 can be configured to stabilize the starting vessel during insertion of introducer 110 or another device or component of system 100.

System 100 can comprise second introducer 130 which can be configured to provide access to a target vessel, such as an artery of the patient when the starting vessel is a vein. In some embodiments, second introducer 130 comprises a 4 French vascular introducer. System 100 can comprise target wire 120 configured to be placed through second introducer 130 and into the target vessel. Target wire 120 can comprise helical section 121 configured to be deployed at the site where the flow pathway is to be created. Helical section 121 can be configured to provide structure and support to the site during a procedure. Additionally, target wire 120 can serve as a visual reference during insertion of vessel-to-vessel guidewire 170, as described herebelow.

System 100 can comprise needle deployment device 140. Needle deployment device 140 can comprise shaft 141 which slidingly receives advanceable crossing needle 145, shown in an advanced state in FIG. 2. Shaft 141 can comprise shaft hub 142 mounted to its proximal end. Shaft 141 can comprise a curved distal portion as shown. Crossing needle 145 can comprise needle hub 146 mounted to its distal end. Movement of needle hub 146 relative to shaft hub 142 can cause crossing needle 145 to advance and retract within shaft 141. Needle hub 146 can be fully advanced toward shaft hub 142 in the configuration of FIG. 2, such that the tip and distal portion of crossing needle 145 is fully advanced out of the distal end of shaft 141.

Crossing needle 145 can comprise a 20 to 24 gauge needle, such as a 22 gauge needle. In some embodiments, the crossing needle comprises a curved distal portion (as shown). The curved distal portions of shaft 141 and/or crossing needle 145 can be aimed at the center of the target vessel prior to insertion into the target vessel. The radius of curvature can be reduced if the clinician has difficulty in aiming the needle tip at the center of the target vessel prior to insertion. Conversely, the radius of curvature can be increased to sufficiently aim the needle tip at the center of the target vessel. Additionally, the crossing needle 145 can comprise a marker, not shown but indicating the direction of curvature. Examples of markers include, but are not limited to: a flat surface; a textured surface; a visualizable marker such as a radiopaque marker; a magnetic marker; an ultrasonic marker or a visible marker; and combinations of one or more of these. In some embodiments, crossing needle comprises a shaped memory alloy, for example, nickel titanium alloy. In some embodiments, shaft hub 142 and/or needle hub 146 comprise a marker or other visible demarcation (e.g. a flat portion) which correlates to the direction of curvature of shaft 141 and/or crossing needle 145, respectively.

System 100 can comprise a guidewire to be placed from the starting vessel to the target vessel, vessel-to-vessel guidewire 170. Guidewire 170 can be configured to be placed via needle deployment device 140. In some embodiments, vessel-to-vessel guidewire 170 comprises a wire with an outer diameter of approximately 0.018″. Vessel-to-vessel guidewire 170 can comprise a marker, not shown but configured to indicate the flow pathway location by positioning of the marker within or otherwise proximate the flow pathway. In some embodiments, vessel-to-vessel guidewire 170 comprises a distal portion and a mid portion. Guidewire 170 mid portion can comprise a different construction than the distal portion. For example, the mid portion of guidewire 170 can be stiffer than the distal portion.

System 100 can comprise clip deployment catheter 150 configured to house and deploy anastomotic clip 160. Clip 160 can comprise a plurality of distal arms 161 and a plurality of proximal arms 162, which can be deployed simultaneously and/or independently. Clip 160 can comprise at least two distal arms 161 and at least two proximal arms 162 configured to deploy and engage the starting vessel and the target vessel. In some embodiments, clip 160 comprises four deployable distal arms 161 and four deployable proximal arms 162. Clip 160 can comprise a shaped memory alloy, such as nickel titanium alloy. In some embodiments, clip 160 is constructed and arranged as described in applicant's U.S. Pat. No. 8,273,095, entitled “Device and Method for Establishing an Artificial Arterio-Venous Fistula”, filed Jul. 13, 2009, the contents of which are incorporated herein by reference in its entirety. Clip 160 can comprise one or more covered portions or otherwise be configured to direct and/or limit blood flow through clip 160, such as is described herebelow in reference to clip 160′ of FIG. 13.

In some embodiments, clip 160 is biodegradable or includes one or more biodegradable portions (e.g. one or more portions of clip are absorbed or otherwise degrade over time). In some embodiments, clip 160 comprises a biodegradable anastomotic device such as is described in applicant's co-pending U.S. Pat. No. 8,926,545, entitled “Device and Method for Establishing an Artificial Arteriovenous Fistula”, filed Apr. 1, 2010, the contents of which are incorporated herein by reference in its entirety.

Clip deployment catheter 150 can comprise shaft 151. Mounted to the proximal end of shaft 151 can be handle 153. On the proximal end of handle 153 can be port 155, which can be operably attached to shaft 151 such that catheter 150 can be advanced over a guidewire (e.g. a guidewire can travel from the distal end of shaft 151 to port 155), such as guidewire 170 after it has been previously placed between a starting vessel and a target vessel as has been described hereabove. Shaft 151 can comprise one or more tubular portions, such as an inner tubular segment that houses clip 160, and an outer tubular segment that covers clip 160 but can be retracted to deploy clip 160, such as is described in applicant's U.S. Pat. No. 8,641,747, entitled “Devices for Arterio-Venous Fistula Creation”, filed Jun. 13, 2005, the contents of which is incorporated herein by reference in its entirety.

Handle 153 can further include control 152 (e.g. a button, slide or lever), where control 152 can be operably configured to allow an operator to deploy distal arms 161 and/or proximal arms 162 of clip 160, such as via retraction of an outer tube or sheath portion of shaft 151 that can be covering one or more portions of clip 160. In some embodiments, a click or other tactile feedback is provided during retraction of a sheath portion of shaft 151. Control 152 can be moved via a stepped or otherwise segmented slot 156. Distal arms 161 can be deployed via moving control 152 from a “first ready to deploy” position to a “first deployed” position which can be achieved by moving control 152 relatively parallel to the longitudinal axis of handle 153. The at least two proximal arms 162 can be queued to be deployed via moving control 152 from the first deployed position to a “second ready to deploy” position. The second ready to deploy position can be achieved by moving control 152 in a direction perpendicular to the longitudinal axis of the handle. Subsequently, proximal arms 162 can be deployed via moving control 152 from the second ready to be deployed position to a “second deployed” position via a motion parallel to the longitudinal axis of the handle. In this embodiment, control 152 can include a safety position comprising a ready to deploy position which can be transitioned by moving control 152 in a direction that is perpendicular to the axis of handle 153. This control advancement arrangement can prevent inadvertent deployment of distal arms 161 and/or proximal arms 162.

In some embodiments, prior to deployment of one or more arms of clip 160, introducer 110 is advanced such that beveled tip 111 applies a force to the wall of the starting vessel. Sufficient force can be applied by introducer 110 to enable an operator to “seat” the starting vessel against the target vessel to assist in proper deployment of clip 160.

In some embodiments, clip deployment catheter 150 can be configured to recapture distal arms 161 and/or proximal arms 162. For example, clip deployment catheter 150 can deploy at least one distal arm 161 and subsequently recapture the at least one distal arm 161.

Clip deployment catheter 150 and/or clip 160 can further comprise at least one marker, not shown but typically a radiopaque and/or ultrasonic marker configured to assist in the rotational positioning of clip 160 at the flow pathway location. For example, the marker can be oriented toward the target vessel prior to deployment of clip 160. In some embodiments, a marker is included on the distal portion of clip deployment catheter 150. In some embodiments, handle 153 comprises one or more markers that are circumferentially aligned with clip 160 prior to its deployment. In some embodiments, clip deployment catheter 150 and/or clip 160 comprise at least one marker configured to longitudinally position clip 160 at the flow pathway location. In these embodiments, the marker can indicate the distal and/or proximal end of clip 160.

Clip deployment catheter 150 can further comprise a projection and/or recess, neither shown but configured to mechanically engage clip 160. The projection and/or pin can be used to stabilize clip 160 within shaft 151, such as when an outer tubular portion of shaft 151 is advanced or retracted.

In some embodiments, clip deployment catheter 150 can be further constructed and arranged to deploy one or more other implants, such as venous stent 195 and/or covered stent 199 described herebelow. Alternatively or additionally, system 100 can comprise a second clip deployment catheter 150 constructed and arranged to deploy venous stent 195 and/or covered stent 199, such as delivery sheath 198 described herebelow in reference to FIG. 11.

System 100 can comprise dilation device 180 configured to dilate clip 160 and/or the flow pathway. Dilation device 180 can include balloon catheter 185, such as a standard angioplasty balloon catheter comprising balloon 186. Attached to the proximal end of balloon catheter 185 can be indeflator 181, typically a standard balloon indeflator device. Alternatively, balloon 186 can comprise a non-balloon expandable such as an expandable cage or radially deployable arms configured to dilate the flow pathway. Balloon catheter 185 can be configured to track over a vessel-to-vessel guidewire, such as guidewire 170 placed between a vein and an artery, such that balloon 186 can be positioned within the flow pathway (e.g. within clip 160). In some embodiments, dilation device 180 expands to a diameter of less than 5 mm, such as to a diameter of approximately 4 mm In some embodiments, a second dilation device 180 is included, such as a device configured to expand to a different diameter than the first dilation device.

System 100 can comprise an agent 187 configured to be delivered by an agent-delivering device, such as when balloon catheter 185 is configured to deliver agent 187. Alternatively or additionally, system 100 can comprise a separate device configured to deliver agent 187. In some embodiments, balloon catheter 185 comprises a first catheter 185 (e.g. a balloon catheter or other radially deployable device) configured to dilate a flow pathway and/or clip 160 as described hereabove, and a second catheter 185 configured to deliver agent 187 to tissue. Catheter 185 can be configured to deliver one or more agents 187, such as one or more agents 187 configured to reduce a current or future stenosis, such as a current or future venous stenosis. An agent-delivering catheter 185 can comprise a balloon 186 comprising a distensible or non-distensible balloon. Catheter 185 can comprise an agent 187 configured as a coating on balloon 186. Alternatively, catheter 185 can be configured to deliver agent 187 from within balloon 186 (e.g. when catheter 185 comprises a porous or otherwise permeable balloon 186). Alternatively or additionally, clip 160 can be configured to deliver agent 187, such as when agent 187 is configured as an eluding coating on clip 160.

Agent 187 of system 100 can comprise one or more agents selected from the group consisting of: an anti-proliferative agent; a chemotherapeutic agent such as paclitaxel; mTOR inhibitors (mammalian target of rapamycin inhibitors), such as Sirolimus or its analogues Zotarolimus or Everolimus; and combinations of one or more of these. Agent 187 can be delivered prior to and/or during a flow pathway creation procedure or a flow pathway modification procedure (such as is described hereabove in reference to FIG. 1), and/or in one or more subsequent procedures. Agent 187 can be delivered to venous wall tissue, such as a full or partial circumferential segment of venous wall tissue proximate (e.g. within 10 cm of) the flow pathway and/or proximate clip 160. In some embodiments, agent 187 is delivered to a 5 mm to 60 mm circumferential segment of venous wall tissue superior to; inferior to; medial to and/or lateral to the flow pathway and/or clip 160. In some embodiments, agent 187 is delivered to a venous wall location in between the flow pathway and the heart (i.e. at a vein location downstream of but within 10 cm of the flow pathway). One or more agents 187 delivered by catheter 185 (and/or another component of system 100) can be configured to prevent or otherwise reduce a stenosis that might otherwise occur in a vein at a location proximate the flow pathway of the present inventive concepts. The delivered agent 187 can be configured to block cell proliferation and associated fibrosis and stenosis. The delivered agent 187 can be configured to be efficacious over an extended time period, such as at least 3 months, at least 6 months, at least 12 months, at least 18 months or at least 24 months. Alternatively, delivered agent 187 can be delivered in multiple procedures, such as to be collectively efficacious for a time period of at least 3 months, at least 6 months, at least 12 months, at least 18 months or at least 24 months. In some embodiments, agent 187 comprises paclitaxel or a similar agent, such as when agent 187 is delivered by anastomotic clip 160 (e.g. agent 187 comprises a coating of clip 160) and/or when agent 187 is delivered by catheter 185 (e.g. when agent 187 comprises a coating on balloon 186 and/or is delivered through a permeable balloon 186). In some embodiments, agent 187 comprises paclitaxel or a similar agent comprising a mass of at least 200 μg (e.g. a mass of at least 200 μg coated onto clip 160 or a mass of between 300 μg and 600 μg coated on and/or positioned within balloon 186 of catheter 185). In some embodiments, at least 80% (e.g. at least 90%) of the included amount of agent 187 is delivered to tissue by balloon 186 and/or clip 160 during a time period of approximately 20 seconds, between 20 seconds and 60 seconds, or approximately 60 seconds. In some embodiments, between 10% to 15% of the amount of agent 187 delivered is still present in the vessel wall 40 minutes after delivery. In some embodiments, catheter 185 and/or balloon 186 are constructed and arranged to deliver agent 187 to tissue over a larger surface area than would correspondingly be delivered if agent 187 comprises a coating of clip 160.

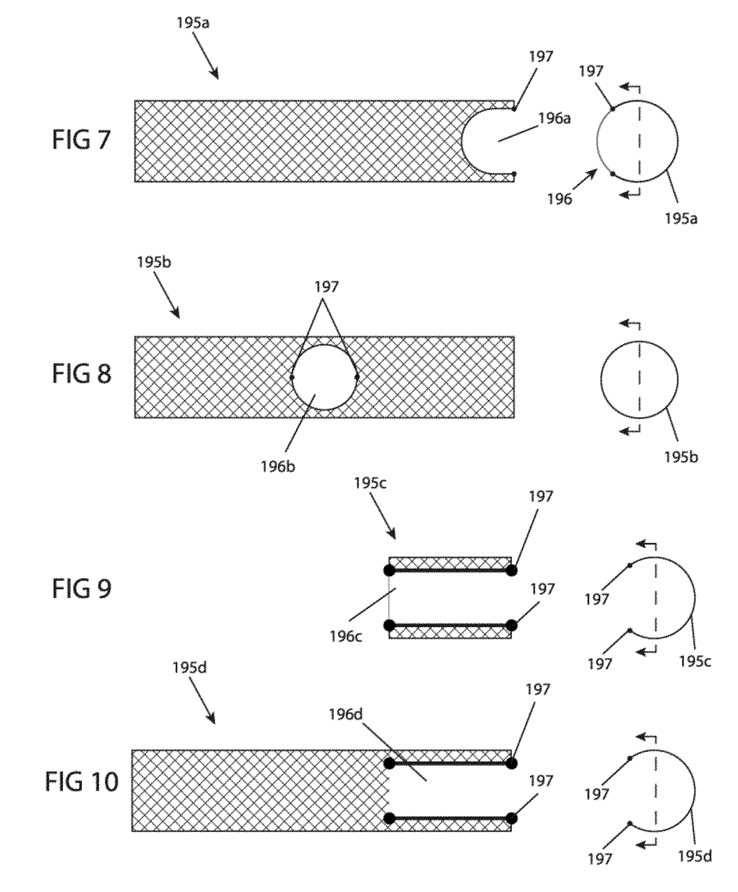

System 100 can include one or more venous stents 195. Stent 195 can comprise a kit of multiple stents with different diameters, such as a set of stents with diameters between 2 mm and 16 mm. One or more stents 195 can comprise a self-expanding stent, a balloon expandable stent, or stents with self-expanding portions and balloon expandable portions. System 100 can include a catheter device for implanting stent 195 into a vein, such as clip deployment catheter 150 and/or delivery sheath 198 described herebelow in reference to FIG. 11. In some embodiments, one or more venous stents 195 comprise an opening for positioning over a flow pathway, such as openings 196a, 196b, 196c or 196d described herebelow in reference to FIG. 7, 8, 9 or 10, respectively.

System 100 can include one or more covered stents 199, such as one or more PTFE covered stents. Stent 199 can be configured to be positioned in a vein or an artery such as to fully or partially occlude a flow pathway of the present inventive concepts as described hereabove in reference to FIG. 1. Covered stent 199 can comprise a kit of multiple stents with different diameters, such as a set of stents with diameters between 2 mm and 10 mm. One or more covered stents 199 can comprise a self-expanding stent, a balloon expandable stent, or stents with self-expanding portions and balloon expandable portions. System 100 can include a catheter device for implanting covered stent 199 into a blood vessel (e.g. in a vein to partially or fully occlude a flow pathway), such as clip deployment catheter 150 and/or delivery sheath 198 described herebelow in reference to FIG. 11.

System 100 can include patient imaging apparatus 190. Non-limiting examples of imaging apparatus 190 include: x-ray; fluoroscope; ultrasound imager; MRI; and combinations of one or more of these Imaging apparatus 190 can allow the clinician to track the movement of all components comprising system 100 as well as view the position of the starting and target vessel relative to each other, as described in detail herein.

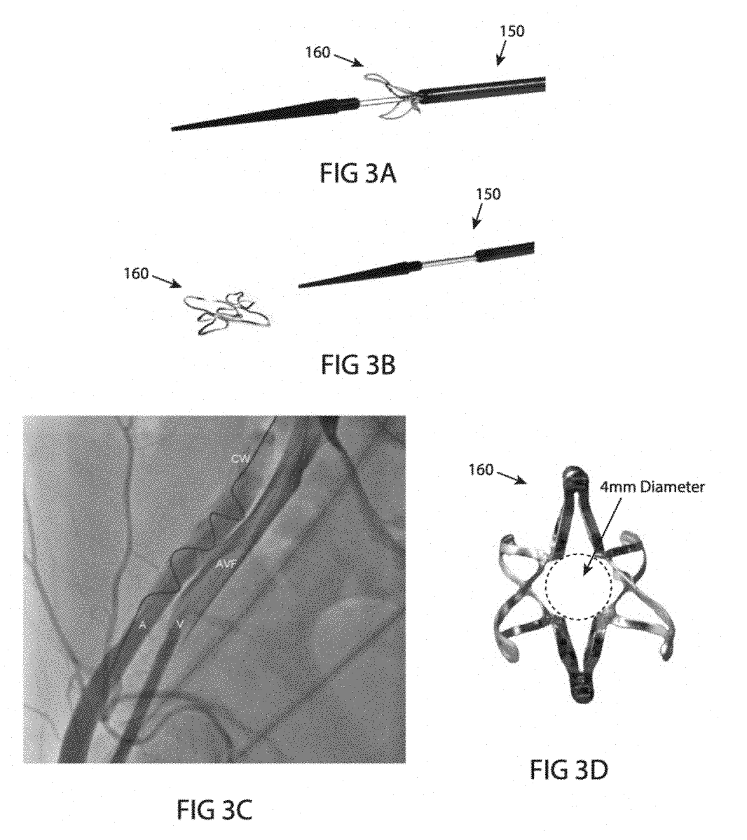

In an exemplary procedure illustrated in FIGS. 3A-D, an anastomotic clip can be deployed to create an iliac arteriovenous flow pathway (e.g. an arteriovenous fistula). Vascular venous and arterial access can be obtained using standard interventional techniques (e.g. through a femoral vein and femoral artery, respectively). FIGS. 3A and 3B illustrate an anastomotic clip delivery device that can be used, including an anastomotic clip that can be implanted. In some embodiments, the anastomotic clip delivery device comprises clip deployment catheter 150, and the anastomotic clip comprises clip 160, each of FIG. 2 hereabove. In FIG. 3C, an angiogram of the flow pathway location AVF is illustrated, including artery A and vein V, prior to flow pathway creation. A radiopaque vessel targeting wire CW, such as target wire 120 of FIG. 2, can be inserted within artery A to provide a radiographic outline of the artery. A venogram can be performed to outline the vein and confirm proximity of the artery and vein at the target crossing location AVF for the creation of the arteriovenous flow pathway. A crossing needle device (e.g. a 22 gauge or 23 gauge crossing needle device), such as needle deployment device 140 of FIG. 2 hereabove, can be placed into vein V, such as a placement over a guidewire and through an introducer device (e.g. introducer 110 of FIG. 2). The crossing needle of the device can be advanced through the wall of vein V into artery A, and a guidewire can be advanced through a lumen of the crossing needle and into artery A. The crossing needle can be subsequently removed and the anastomotic clip deployment catheter 150 can be tracked across the puncture site. The anastomotic clip can then be deployed so that the expanded arms of the anastomotic clip attach to the inner walls of artery A and vein V, and the retention arms maintain the anastomotic clip in the proper position (deployed position shown in FIG. 3D). After removal of the delivery system, a balloon catheter (e.g. a 2.5-4.5 mm diameter balloon catheter) can be inserted into the center of the anastomotic clip and inflated to expand the anastomotic clip to a target diameter as described herein. The balloon can then be deflated and removed. An angiogram can confirm the patency of the flow pathway.

Referring now to FIG. 4, a flow chart of a method of creating a flow pathway between a starting vessel and a target vessel at a flow pathway location, consistent with the present inventive concepts is illustrated. In Step 410, a procedural planning assessment of a patient is performed. Step 420 comprises placing a first introducer into a starting vessel (e.g. a vein), and placing a second introducer into a target vessel (e.g. an artery). In Step 430, an angiographic orientation is performed and a flow pathway location is selected. Step 440 comprises placing a vessel-to-vessel guidewire between the vein and the artery. Step 450 comprises placing an anastomotic clip at the flow pathway location. In some embodiments, system 100 and/or one or more components of system 100 of FIG. 2 are used to perform the method of FIG. 4.

The starting vessel can comprise a vein, and can be selected from the group consisting of: inferior vena cava (IVC); saphenous; femoral; iliac; popliteal; brachial; basilic; cephalic; medial forearm; medial cubital; axillary; and jugular. The target vessel can comprise an artery, and can be selected from the group consisting of: aorta; axillary; brachial; ulnar; radial; profundal; femoral; iliac; popliteal and carotid. In a preferred embodiment, the starting vessel and target vessel can comprise an external iliac. In an alternate embodiment, the starting vessel can comprise an artery and the target vessel can comprise a vein.

In Step 410, the first step in the illustrated method of the present inventive concepts comprises procedural planning. This step comprises properly orienting the vein and the artery, meaning a clinician becomes familiar with the anatomical orientation of the vein and artery relative to each other. Understanding the orientation of the vessels with respect to one another can be achieved through analysis of one or more images provided by an imaging apparatus (e.g. a fluoroscope) such as imaging apparatus 190 of FIG. 2. In some embodiments, at least one of the vein or artery has a diameter of at least 5 mm proximate the flow pathway location. In another embodiment, both the vein and artery have a diameter of at least 5 mm proximate the flow pathway location.

In Step 420, the method comprises placing a first introducer into the vein. The first introducer can comprise an approximately 11 French introducer having a beveled tip, such as introducer 110 of FIG. 2 described hereabove. In some instances, the beveled tip of the first introducer can be rotated during insertion into the vein. Rotation of the introducer can be helpful during insertion into the starting vessel due to the tendency of the beveled tip to lift and pull back. Additionally or alternatively, the introducer can be vibrated while it is advanced into the vein, such as to ease insertion. Step 420 can further comprise pre-dilating the vein with a dilator, such as a 13 French dilator, prior to placing the introducer into the vein. Additionally, a second introducer can be placed into the artery. The second introducer can comprise a 4 French introducer, such as second introducer 130 described in FIG. 2 hereabove. The method further comprises placing a target wire into the second introducer and then into the artery such that the distal end of the target wire is positioned 5 cm to 10 cm past the flow pathway location, and configured to serve as a visual reference to a clinician. The target wire, typically including a helical section, is advanced. The advancement can be combined with retracting the introducer such that the helical section of the wire is deployed at the targeted flow pathway location.

In Step 430, the method comprises performing angiographic orientation and selecting a flow pathway location. Choosing the flow pathway location can be based upon a lack of thrombus or other soft tissue occlusive matter at the vascular location, as well as lack of plaque or calcified matter. The flow pathway location can be chosen at a location where the vein is less than or equal to 3 mm apart from the artery. Techniques can be used to image the vein and artery in side-by-side configurations as well as overlapping (i.e. on top of each other in the image) orientations. Rotation of the imaging apparatus (e.g. fluoroscope) by an angular displacement of 90° can modify the provided image from a side-by-side image to an overlapping image, and back again. In some embodiments, after a flow pathway location has been selected, a clinician can orient the imaging apparatus such that the vein and artery are shown overlapping, such as with the vein on top of the artery. In some embodiments, the clinician can position a fluoroscope or other imaging apparatus at an angle to the patient approximating 35° RAO.

In Step 440, the method comprises placing a vessel-to-vessel guidewire first into the vein (only), such as while the vein and artery are being imaged in an overlapping orientation, as described in Step 430 hereabove. In some embodiments, the vessel-to-vessel guidewire can be placed into the vein through a dilator. A next step comprises placing a needle delivery device over the vessel-to-vessel guidewire and into the vein. The needle delivery device can comprise a marker, such as is described in FIG. 2 hereabove, such that a clinician can orient the marker (e.g. and also the needle) toward the artery. The guidewire can be retracted, and subsequently the needle of the needle delivery device can be advanced toward the target wire and toward the artery.

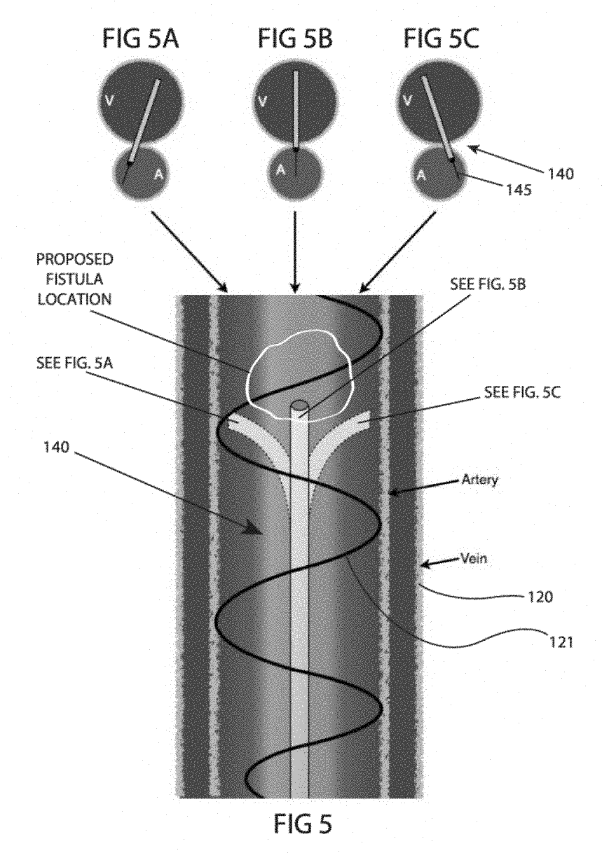

Prior to inserting the crossing needle into the artery, a clinician can aim the needle tip at the center of the artery to ensure desired engagement of the artery with the needle, such as by rotating the proximal end of the needle or a device containing the needle. In some embodiments, the needle or needle delivery device includes a proximal hub with a demarcation (e.g. a flat portion or other marker) positioned to indicate the orientation of a curved distal portion of the needle, such as is described in reference to needle deployment device 140 of FIG. 2 hereabove. In this operation, a clinician can torque or otherwise rotate the needle such that the direction of the needle curvature can be distinguished on the imaging apparatus (e.g. fluoroscope). Confirming the direction of needle curvature ensures that the needle is to be advanced in the desired direction, such as into the center of the artery. In some embodiments, a target wire is placed in the target vessel, such as target wire 120 of FIG. 2 described hereabove. The needle can comprise a curved tip, and the radius of curvature can be reduced (e g manually reshaped) if a clinician has difficulty in aiming the needle at the center of the target vessel prior to insertion. Conversely, the radius of curvature can be increased to sufficiently aim the needle tip at the center of the target vessel. In some embodiments, the needle delivery catheter is oriented using the techniques described herebelow in reference to FIG. 5.

In some embodiments, a clinician confirms that the distal portion of the vessel-to-vessel guidewire is located within the lumen of the artery. Alternatively or additionally, a clinician confirms the vessel-to-vessel guidewire is parallel with the target wire previously placed in the artery. In some embodiments, a clinician confirms that the needle is positioned within the target vessel by using a dye injection through the needle. Alternatively or additionally, a clinician can confirm that the needle is properly positioned in a target vessel by measuring the pressure in a distal portion of the needle, such as to confirm presence in an artery by confirming arterial pressure is recorded.

While the above method describes a vein-to-artery advancement of the vessel-to-vessel guidewire and subsequent devices, in some embodiments an artery-to-vein approach is performed. In these embodiments, the vessel-to-vessel guidewire and needle deployment device are placed first into the artery (as described hereabove in reference to the vein), and subsequently the vessel-to-vessel guidewire is advanced from the artery into the vein via the crossing needle. In these embodiments, the anastomotic clip deployment catheter and/or any other flow pathway modification devices (e.g. a dilating balloon) can also be advanced from artery to vein.

In Step 450, the method comprises placing an anastomotic clip at a flow pathway location. Prior to performing Step 450, a clinician can retract the crossing needle while maintaining the position of the target wire. Next, the target wire can be removed from the second introducer. The target wire can also be removed after Step 450.

In Step 450, a clinician can position the vein and artery such that the vein and artery are shown slightly apart from each other on the image (e.g. not overlapping). In one embodiment, this can be achieved by further rotating an imaging apparatus (e.g. a fluoroscopy unit) with an angular rotation between 45° to 90° after an overlapping image is obtained (e.g. an image obtained during a dual contrast injection of both the artery and vein).

Next, the tip of the clip deployment catheter (with a pre-loaded anastomotic clip) can be placed at the flow pathway site. In this step, a clinician can apply forward pressure and rotate the clip deployment catheter. The clip can comprise at least two distal arms and at least two proximal arms that can be deployed simultaneously or independently via a control located on the handle of the catheter (such as is described hereabove).

Step 450 further comprises deploying the anastomotic clip in the flow pathway, such as is described in detail in reference to clip deployment catheter 150 of FIG. 2 hereabove. The clip distal arms are deployed by moving a control on the clip deployment catheter from a ready to deploy position to a first deployed position, which can be achieved by moving the control relatively parallel to the longitudinal axis of the handle. Prior to deploying the proximal arms of the clip, a clinician can retract the first introducer to the flow pathway location and seat the vein against the artery. The clip deployment catheter can comprise a marker located on its distal end. Using this marker, a clinician can pull the clip deployment catheter back such that the marker is aligned with the distal end of the first introducer.

In a next operation of STEP 450, the proximal arms can be queued to be deployed via moving the control from the first deployed position to a second ready to deploy position. The ready to deploy position can be achieved by moving the control in a direction perpendicular to the longitudinal axis of the handle. Subsequently, the proximal arms can be deployed via moving the control from the second ready to be deployed position to the second deployed position via a motion parallel to the longitudinal axis of the handle. In this embodiment, the control includes a safety position comprising a ready to deploy position which can be transitioned to a subsequent position by moving the control in a direction that is perpendicular to the axis of the handle. This control arrangement can prevent inadvertent deployment of the distal and/or proximal arms. After deployment of the proximal arms, a clinician can retract the first introducer from the anastomosis site, such as a retraction of approximately 2 cm to 3 cm, followed by retracting the clip deployment catheter.

The method can further comprise dilating the flow pathway via a balloon or other expandable member. For example, a clinician can track a balloon catheter over the target wire and inflate the balloon. In a typical embodiment, the balloon catheter comprises a diameter of 4 mm to 5 mm and can be inflated via a 4 mm by 1.5 cm non-conforming balloon and indeflator device. The balloon then can be deflated and retracted out of the implant.

The method can further comprise verifying patency of the anastomotic clip. This verification can be achieved via a contrast/saline solution injected into the second introducer. A clinician can then remove all devices once it is confirmed that the clip is positioned as desired.

The method can further comprise placing a second anastomotic clip, such as a second anastomotic clip 160 of FIG. 2 described hereabove. Alternatively or additionally, the method can further comprise creating a second flow pathway, such as a second flow pathway created during the same clinical procedure or a subsequent clinical procedure. The second flow pathway can be between the same two vascular locations as the first flow pathway, or one or both of the second flow pathway vascular locations can be different (e.g. a different vein and/or artery).

Referring now to FIG. 5, an angiographic view of a patient's vein and artery prior to advancement of a needle into the artery is illustrated, such as can be performed in Step 440 of the method of FIG. 4 described hereabove, consistent with the present inventive concepts. In the illustrated embodiment, a clinician has oriented an imaging apparatus (e.g. a fluoroscope or other imaging apparatus 190 of FIG. 2), such that the segments of vein and artery at a proposed flow pathway location are overlapping (i.e. on top of each other in the image). The clinician has placed a target wire 120 into a patient's artery such that the helical section 121 can be positioned at the proposed flow pathway location. Additionally, needle deployment device 140 has been advanced intraluminally through the vein, as shown, such that its distal end is proximate the proposed flow pathway location. A next step can comprise advancing needle 145 toward the helical section 121 at the proposed flow pathway location.

Prior to insertion of needle 145 into the artery, a clinician can rotate needle deployment device 140 such that the direction of the needle deployment device 140 curvature can be viewed (e.g. a non-linear, curved segment can be visualized) on the imaging apparatus. Confirming the direction of curvature ensures that needle 145 can be advanced in the desired direction, such as into the center of the artery. For example, if a clinician rotates needle deployment device 140 such that its tip is positioned as shown in FIG. 5A or 5C, a clinician will be aiming to an off-center location of the patient's artery. If a clinician rotates needle deployment device 140 such that its tip is positioned as shown if FIG. 5B, needle 145 will subsequently be advanced into the relative center of the patient's artery. The radius of curvature of a needle deployment device 140 can be reduced (e.g. by manual reshaping or by selecting a different needle deployment device 140) if a clinician has difficulty in aiming needle 145 at the center of the artery prior to insertion. Conversely, the radius of curvature of needle deployment device 140 can be increased to create a more desirable needle 145 advancement trajectory.

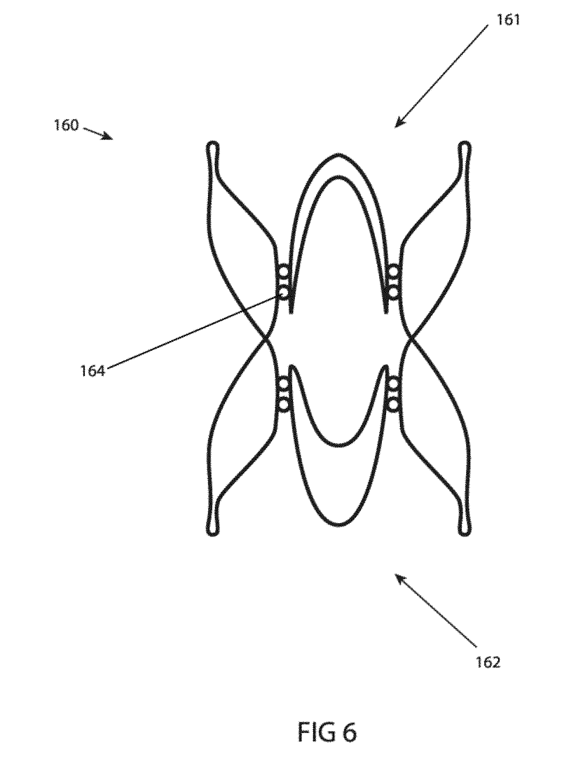

Referring now to FIG. 6, a perspective view of an anastomotic clip is illustrated, consistent with the present inventive concepts. Clip 160 can comprise at least two distal arms 161 and at least two proximal arms 162. In the illustrated embodiment, clip 160 comprises four distal arms 161 and four proximal arms 162. In some embodiments, clip 160 is constructed and arranged as described in applicant's U.S. Pat. No. 8,273,095, entitled “Device and Method for Establishing an Artificial Arterio-Venous Fistula”, filed Jul. 13, 2009, the contents of which are incorporated herein by reference in its entirety

Clip 160 can be formed from a single tube of resilient material, such as nickel titanium alloy, spring steel, glass or carbon composites or polymers, or a pseudoelastic (at body temperature) material such as nickel titanium alloy or comparable alloys and polymers. Clip 160 can be formed by laser cutting several closed-ended slots along the length of a tube (leaving the extreme distal and proximal edges of the tube intact) and cutting open-ended slots from the longitudinal center of the tube through the distal and proximal edges of the tube. The open-ended slots can be cut between each pair of closed-end slots to form a number of loops joined at the center section by waist segments. Many other fabrication techniques can be utilized, for example, clip 160 can be made of several loops of wire welded together at a waist section.

After a tube is cut as described above, it can be formed into its eventual resiliently expanded configuration. In this configuration, the loops turn radially outwardly from the center section, and evert toward the center plane of the center section, thus forming clinch members (i.e. distal arms 161 and proximal arms 162), in the form of arcuate, everted, petaloid frames at either end of the loop, extending from the generally tubular center section formed by waist segments. For clarity, the term everted is used here to mean that the arc over which the petaloid frame runs is such that the inside surface of clip 160 faces radially outwardly from the cylinder established by the tube.

Once clip 160 has resiliently expanded to the extent possible given its impingement upon the walls of the starting vessel and the target vessel, the center section can be further expanded by plastic deformation of clip 160 and/or temporary or permanent deformation of tissue surrounding the flow pathway. This expansion can be accomplished by inflating a balloon, not shown, within the center section of clip 160 and the flow pathway, such as to expand the center section of clip 160 beyond its elastic or superelastic deformation range (and/or to deform the surrounding tissue). By plastically deforming the center section of clip 160, the center section becomes more rigid and able to withstand the compressive force of the walls of the starting and target vessels.

As illustrated, the construction provides several pairs of longitudinally opposed (that is, they bend to come into close proximity to each other, and perhaps but not necessarily, touch) and aligned (they are disposed along the same longitudinal line) distal arms 161 and proximal arms 162. Overall, the petaloid frames of distal arms 161 form a “corolla,” analogous to the corolla of a flower, flange or rivet clinch, which impinges on the starting vessel wall and prevents expulsion into the target vessel, and the petaloid frames of proximal arms 162 form a corolla, flange or rivet clinch (this clinch would be analogous to a rivet head, but it is formed like the clinch after insertion of the rivet), which impinges on the target vessel wall and prevents the expulsion of clip 160 into the target vessel. Also, the central section forms a short length of rigid tubing to keep the flow pathway open. The resilient apposition of the at least two distal arms 161 and at least two proximal arms 162 will securely hold clip 160 in place by resiliently clamping the walls of the starting vessel and the target vessel, even over a considerable range of wall thickness or “grip range.”

The respective lengths of arms 161 and 162 can be variably sized to maximize or optimize the stability of clip 160 with respect to the vessels when deployed between adjacent vessels. Moreover, varying the lengths of the respective arms can further provide additional advantages. For instance, the arms which are shortened in length can facilitate the positioning and securement of clip 160 between the vessels by allowing for the relatively shorter member to swing into position within the vessel lumen during deployment, as described in further detail below. Additionally, a shorter member can provide for a minimized implant size when placed against the vessel interior wall for securement as well as mitigating any physiologic reaction to the implant, e.g., a reduction in thrombosis, etc. Additionally, arms 161 and/or 162 which are lengthened relative to other arms can provide for increased clip stability by increasing the amount of force applied against the tissue walls.

Moreover, arms having different lengths can additionally place the adjacent vessels in tension such that the vessel walls are drawn towards one another and arms 161 and/or 162 contact the vessel luminal walls to stabilize not only clip 160 within the vessels but also the vessels with respect to one another. Additionally, having one or more arms, such as distal arms 161, sized to have a length shorter than its respective apposed clinch member can also facilitate the deployment and/or positioning of distal arms 161 within the vessel since the shorter length clinch members can more easily “swing” through an arc within the vessel lumen without contacting the interior walls. Arms with differing lengths can further be configured to align along different planes when deployed to facilitate vessel separation, if so desired.

Clip 160 can further comprise at least one marker, not shown, configured to rotationally position the clip at the flow pathway location. For example, a marker can be oriented toward the target vessel prior to deployment of clip 160. Alternatively or additionally, a marker can be oriented based upon a patient image (e.g. a real-time fluoroscopy image). In yet another embodiment, clip 160 can comprise at least one marker configured to longitudinally position the clip 160 at the flow pathway location. A marker can indicate the distal and/or proximal end of clip 160.

Clip 160 can further comprise holes 164 configured to engage a projection of a clip deployment catheter, such as to allow the shaft of the clip deployment catheter, not shown, to be retracted while clip 160 remains positioned in the distal portion of the shaft. In one embodiment, holes 164 are constructed and arranged about the clip asymmetrically such that clip 160 can be attached in the proper orientation.

FIGS. 7-10 illustrate various embodiments of a venous stent 195 for implanting in a vein proximate a flow pathway of the present inventive concepts. The venous stent can be constructed and arranged to treat and/or prevent a venous stenosis, such as a venous stenosis that might be present or otherwise (without the presence of venous stent 195) form at a location up to 3 cm from an anastomotic clip (e.g. clip 160 described herein) or a flow pathway created without an anastomotic clip (e.g. a flow pathway created through delivery of RF or other energy). Venous stent 195 of FIGS. 7-10 can comprise a self-expanding and/or balloon expandable structure. Venous stent 195 can comprise a cutaway section that is positioned over the venous entry portion of the flow pathway, such as to avoid venous stent 195 impeding blood flow through the flow passageway. Venous stent 195 can be constructed and arranged to shield a segment of the vein proximate the flow pathway from the turbulent flow caused by blood entering from the connected artery. In some embodiments, venous stent 195 comprises at least one opening 196, such as an opening 196 constructed and arranged to be positioned over the flow pathway. Venous stent 195 can comprise one or more radiopaque markers, such as markers 197 described herebelow, such as a marker positioned to identify the cutaway portion of venous stent 195 or any opening along the length of stent 195 configured to accommodate blood flow through a flow pathway of the present inventive concepts. System 100 of FIG. 2 can comprise one or more venous stents 195, such as a kit of venous stents 195 with different parameters, such as one or more different parameters selected from the group consisting of: length; cutaway dimension; cutaway location relative to an end of stent 195; diameter; pattern (e.g. pattern of stent mesh); number of radiopaque markers; position of a radiopaque marker; tensile strength; hoop strength; and combinations of one or more of these. In some embodiments, stent 195 comprises a first stent 195 with a cutaway portion, and a second stent 195 without a cutaway portion, such as when the first stent 195 comprises a C-shaped cutaway portion on one end, and the second stent 195 is configured to be positioned proximate the C-shaped cutaway portion. In some embodiments, clip 160 is configured to mechanically engage venous stent 195, such as to frictionally engage a portion of venous stent 195.

Referring now to FIG. 7, a side and end view of a venous stent including a cutaway end is illustrated, consistent with the present inventive concepts. Stent 195a comprises a C-shaped opening 196a positioned on one end of stent 195a as shown. Stent 195a can comprise a self-expanding stent. Alternatively or additionally, stent 195a can comprise at least a balloon expandable portion. Opening 196a is configured to be positioned in a vein proximate (e.g. positioned over) a flow pathway of the present inventive concepts. Stent 195a comprises radiopaque marker 197 positioned along at least a portion of the periphery of opening 196a. Stent 195a can be constructed and arranged to be implanted by a delivery sheath, such as delivery sheath 198 described herebelow in reference to FIG. 11.

Referring now to FIG. 8, a side and end view of a venous stent including an opening along its length is illustrated, consistent with the present inventive concepts. Stent 195b comprises a circle-shaped opening 196b positioned along a mid-portion of stent 195b as shown. Stent 195b can comprise a self-expanding stent. Alternatively or additionally, stent 195b can comprise at least a balloon expandable portion. Opening 196b is configured to be positioned in a vein proximate (e.g. positioned over) a flow pathway of the present inventive concepts. Stent 195b comprises radiopaque marker 197 positioned along at least a portion of the periphery of opening 196b. Stent 195b can be constructed and arranged to be implanted by a delivery sheath, such as delivery sheath 198 described herebelow in reference to FIG. 11.

Referring now to FIG. 9, a side and end view of a venous stent including a C-shaped profile is illustrated, consistent with the present inventive concepts. Stent 195c comprises a rectangular-shaped opening 196c positioned along the entire length of stent 195c as shown. Stent 195c can comprise a self-expanding stent. Alternatively or additionally, stent 195c can comprise at least a balloon expandable portion. Opening 196c is configured to be positioned in a vein proximate (e.g. positioned over) a flow pathway of the present inventive concepts. Stent 195c comprises radiopaque marker 197 positioned along at least a portion of the periphery of opening 196c. Stent 195c can be constructed and arranged to be implanted by a delivery sheath, such as delivery sheath 198 described herebelow in reference to FIG. 11.

Referring now to FIG. 10, a side and end view of a venous stent including a C-shaped end portion is illustrated, consistent with the present inventive concepts. Stent 195d comprises a rectangular-shaped opening 196d positioned along an end portion of stent 195d as shown. Stent 195d can comprise a self-expanding stent. Alternatively or additionally, stent 195d can comprise at least a balloon expandable portion. Opening 196d is configured to be positioned in a vein proximate (e.g. positioned over) a flow pathway of the present inventive concepts. Stent 195d comprises radiopaque marker 197 positioned along at least a portion of the periphery of opening 196d. Stent 195d can be constructed and arranged to be implanted by a delivery sheath, such as delivery sheath 198 described herebelow in reference to FIG. 11.

Referring now to FIG. 11, a side view of a venous stent being deployed from a delivery sheath is illustrated, consistent with the present inventive concepts. Delivery sheath 198 comprises an elongate shaft comprising a lumen through which a venous stent, such as venous stent 195c shown and described hereabove in reference to FIG. 9, can be delivered (e.g. pushed out via a delivery rod and/or unsheathed by pulling back the elongate shaft surrounding venous stent 195c). In some embodiments, delivery sheath 198 is of similar construction and arrangement to clip deployment catheter 150 described herein.

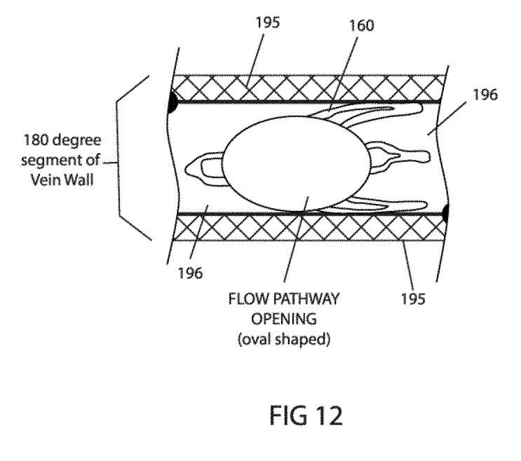

Referring now to FIG. 12, a side sectional, anatomical view of an anastomotic clip and venous stent positioned in and near to but away from, respectively, a flow pathway is illustrated, consistent with the present inventive concepts. Anastomotic clip 160 has been implanted in a flow pathway as described hereabove (e.g. a flow pathway with an oval shaped cross section as shown). Venous stent 195 comprising rectangular shaped opening 196 along the length of stent 195 (e.g. rectangular shaped opening 196c shown in FIG. 9 or rectangular-shaped opening 196d shown in FIG. 10) has been implanted such that opening 196 is aligned with the flow pathway opening in the vein wall (e.g. such that stent 195 provides a stenting force to the vein without obstructing blood flow through the flow pathway). In some embodiments, venous stent 195 comprises an oval shaped opening for positioning over a flow pathway of the present inventive concepts.

Referring now to FIG. 13 is a side sectional, anatomical view of an anastomotic clip including a covered portion and positioned in a flow pathway is illustrated, consistent with the present inventive concepts. Anastomotic clip 160′ has been implanted in a flow pathway as described hereabove. Anastomotic clip 160′ comprises covered portion 165 which is positioned on a venous portion of clip 160′. Covered portion 165 can be constructed and arranged to direct the flow of blood in the vein in a single direction (e.g. to the right of the page as shown in FIG. 13). Covered portion 165 can be positioned to cause blood to flow in the vein in a retrograde direction (e.g. in a revascularization procedure) or in a normal venous return flow direction (e.g. when anastomotic clip 160′ is implanted to reduce systemic vascular resistance and/or to enhance long term patency of a venous stent implanted to treat a venous stenosis as described herebelow in reference to FIG. 14, 15 or 16). Covered portion 165 can comprise a material selected from the group consisting of: PTFE; nickel titanium alloy; polyurethane; one or more bioerodible polymers; a woven mesh of biocompatible polymers and/or nickel titanium fibers; and combinations of one or more of these.

Referring now to FIG. 13A, a side sectional, anatomical view of the flow pathway of FIG. 13 is illustrated, showing only the covered portion of the anastomotic clip, consistent with the present inventive concepts. Covered portion 165 is oriented to direct blood passing through the flow pathway in a single direction (to the right of the page as shown).

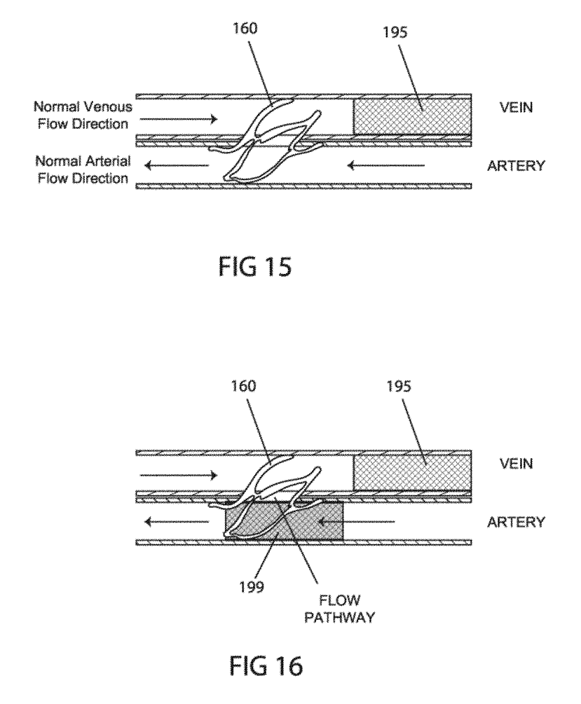

Referring now to FIGS. 14, 15 and 16, a system and a method for treating a venous stenosis is illustrated, consistent with the present inventive concepts. FIG. 14 illustrates a flow chart of a method of treating a venous occlusion. FIG. 15 illustrates a side sectional, anatomical view of a system including a venous stent positioned at a venous stenosis and an anastomotic clip positioned in a flow pathway. FIG. 16 illustrates a side sectional, anatomical view of the system of FIG. 15, further including a covered stent positioned in the vein to occlude the flow pathway. In STEP 1410, a stent, such as venous stent 195 shown in FIGS. 15 and 16 and described hereabove in reference to FIG. 2, is implanted in a vein, such as to treat and/or prevent a venous stenosis as has been described herein.

In STEP 1420, a flow pathway of the present inventive concepts is created, such as when an anastomotic clip is positioned in the flow pathway, such as anastomotic clip 160 shown in FIGS. 15 and 16 and described hereabove in reference to FIG. 2.

In STEP 1430, a flow pathway assessment can be performed, such as to analyze (e.g. quantify) flow through the flow pathway and/or flow through venous stent 195.

In STEP 1440, a flow pathway modification can be performed and/or a second flow pathway can be created. Flow pathway modification can include dilating the flow pathway (e.g. to increase flow), such as has been described hereabove in reference to FIG. 1 or 2. Flow pathway modification can include reducing the flow pathway (e.g. to decrease flow), also as has been described hereabove.

In STEP 1450, a period of time is allowed to elapse, such as to allow flow stabilization within venous stent 195 (and/or the flow pathway) and/or remodeling of tissue proximate venous stent 195 and/or anastomotic clip 160. The wait time of STEP 1450 can comprise at least one hour, at least 1 week, at least 1 month, at least 3 months or at least 6 months after the flow pathway creation procedure of STEP 1420.

In STEP 1460, a patient assessment procedure is performed, such as to assess flow within venous stent 195 and/or the flow pathway, and/or to assess tissue remodeling proximate venous stent 195 and/or the flow pathway. Based on the patient assessment, additional waiting time can be included in STEP 1460.

After sufficient wait time has been determined or confirmed, STEP 1470 is performed in which the flow pathway is fully or at least partially occluded, such as by placing a covered stent in the artery or vein to cover at least a portion of an end of the flow pathway (at least partially cover an end of the flow pathway at the venous wall or the arterial wall). For example, as shown in FIG. 16, covered stent 199 can be implanted in the artery and positioned to fully occlude the flow pathway created using anastomotic clip 160.

While the preferred embodiments of the devices and methods have been described in reference to the environment in which they were developed, they are merely illustrative of the principles of the inventions. Modification or combinations of the above-described assemblies, other embodiments, configurations, and methods for carrying out the invention, and variations of aspects of the invention that are obvious to those of skill in the art are intended to be within the scope of the claims. In addition, where this application has listed the steps of a method or procedure in a specific order, it may be possible, or even expedient in certain circumstances, to change the order in which some steps are performed, and it is intended that the particular steps of the method or procedure claim set forth herebelow not be construed as being order-specific unless such order specificity is expressly stated in the claim.

What is claimed:

1. A system for creating an arteriovenous flow pathway in a patient, comprising:
a vessel-to-vessel guidewire;
a needle delivery device constructed and arranged to place the vessel-to-vessel guidewire from a starting vessel to a target vessel;
a flow creation device constructed and arranged to be advanced over the vessel-to-vessel guidewire and to create a flow pathway between the starting vessel and the target vessel; and
a stent for positioning in a vessel;
wherein at least one of the starting vessel or the target vessel is a vein, and the other of the starting vessel or the target vessel is an artery;
wherein the stent is constructed and arranged to be positioned in the vein and to at least one of prevent or treat venous stenosis.

2. The system according to claim 1, wherein the system is constructed and arranged to treat a patient disease or disorder selected from the group consisting of: angina; angina pectoris; mitral regurgitation; venous stenosis; deep vein thrombosis; hypertension; arterial hypertension; chronic obstructive pulmonary disease; congestive heart failure; lung fibrosis; adult respiratory distress syndrome; lymphangioleiomyomatosis; pulmonary hypertension; sleep apnea such as sleep apnea due to hypoxemia or hypertension; and combinations thereof.

3. The system according to claim 1, wherein the vessel-to-vessel guidewire comprises a marker.

4. The system according to claim 1, wherein the needle delivery device comprises an advanceable needle.

5. The system according to claim 1, wherein the needle delivery device comprises a curved needle.

6. The system according to claim 1, wherein the flow creation device comprises a balloon catheter configured to dilate tissue positioned between the first vascular location and the second vascular location.

7. The system according to claim 1, wherein the flow creation device comprises an energy delivery device constructed and arranged to deliver energy to tissue positioned between the first vascular location and the second vascular location.

8. The system according to claim 1, wherein the flow creation device comprises a clip deployment catheter comprising an anastomotic clip.

9. The system according to claim 8, wherein at least one of the clip deployment catheter or the clip comprises at least one marker constructed and arranged to rotationally position the clip.

10. The system according to claim 8, wherein the clip comprises multiple deployable arms, and wherein the clip deployment catheter is constructed and arranged to deploy at least one of the deployable arms and subsequently recapture the one of the deployable arms.

11. The system according to claim 1, further comprising a target wire constructed and arranged for positioning in the target vessel.

12. The system according to claim 11, wherein the target wire comprises a helical distal portion.

13. The system according to claim 11, wherein the target wire comprises a radiopaque distal portion.

14. The system according to claim 1, wherein the stent comprises a self-expanding stent.

15. The system according to claim 1, wherein the stent comprises a balloon expandable stent.

16. The system according to claim 1, wherein the stent comprises multiple stents.

17. The system according to claim 1, wherein the stent comprises an opening positioned along the length of the stent.

18. The system according to claim 1, further comprising an agent configured to reduce a venous stenosis.

19. The system according to claim 1, further comprising an agent delivery catheter configured to deliver an agent to a vessel wall.

20. The system according to claim 19, wherein the agent delivery catheter is constructed and arranged to reduce a future vessel stenosis.

21. The system according to claim 19, wherein the agent delivery catheter comprises a balloon.

22. The system according to claim 21, wherein the agent delivery catheter further comprises a coating on the balloon comprising the agent to be delivered to the vessel wall.

23. The system according to claim 21, wherein the balloon comprises a permeable balloon constructed and arranged to allow agent to pass therethrough and into the vessel wall.
